(19) United States
(12) Patent Application Publication
(10) Pub. No.: US 2003/0023191 A1
(43) Pub. Date: Jan. 30, 2003

(54) Method and apparatus for producing oscillating signals representing tremor, for filtering the signals, and for generating interpretations of the data to diagnose conditions associated with the tremor

(76) Inventor: Robert M. Tripp, Fountain Hills, AZ (US)

Correspondence Address: Tod R. Nissle, Esq., TOD R. NISSLE, P.C., P.O. Box 55630, Phoenix, AZ, 85078, US

(21) Appl. No.: 09/839,005

(22) Filed: Apr. 20, 2001

(51) Int. Cl. A61B 5/103; A61B 5/117

(52) U.S. Cl. 600/595

(57) ABSTRACT

An apparatus and method are provided for producing, filtering, and evaluating a cyclical signal representing tremor in a patient. The cyclical signals can be utilized to generate data which assist in identifying a condition that causes or is associated with tremor.

This invention was made with government support under Grant No. R44 MH54927 awarded by the National Institute of Health. The government has certain rights in the invention.

This invention relates to a method and apparatus for measuring and evaluating tremor.

More particularly, this invention relates to a method and apparatus for producing, filtering, and evaluating a cyclical signal representing tremor in a patient.

In a further respect, the invention relates to a method and apparatus for evaluating cyclical measurement signals produced by tremor so that the cyclical signals can be utilized to determine a condition which causes or is associated with the tremor.

Apparatus for measuring tremor is well known. An accelerometer is one instrument utilized to produce tremor measurements. Other apparatus can be utilized to measure tremor. While measurement of tremor has long been accomplished, the measurements which are currently made apparently do not clearly identify the tremor component and can not be readily utilized to determine with accuracy conditions which are associated with the tremor experienced by a patient.

Accordingly, it would be highly desirable to provide an improved method and apparatus which would more clearly identify the tremor component and which would enable a condition associated with tremor to be diagnosed with greater accuracy.

Therefore, it is a principal object of the invention to provide an improved method and apparatus for generating tremor measurements.

A further object of the instant invention is to provide an improved method and apparatus for determining the condition associated with a patient's tremor.

These and other, further and more specific objects and advantages of the invention will be apparent to those skilled in the art from the following detailed description thereof, taken in conjunction with the drawings, in which:

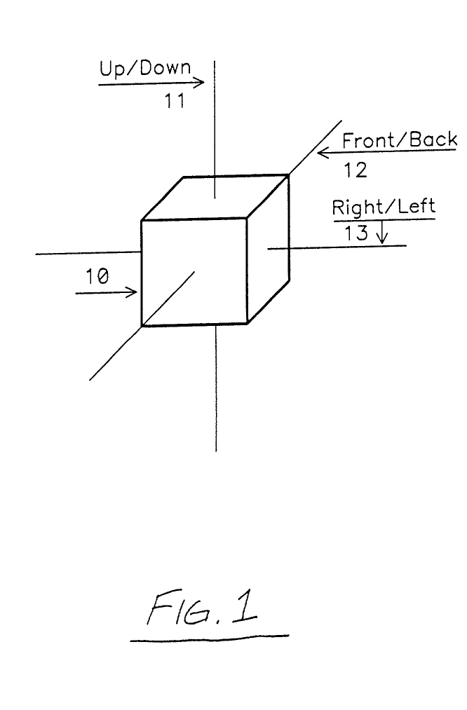

FIG. 1 is a perspective view illustrating an accelerometer;

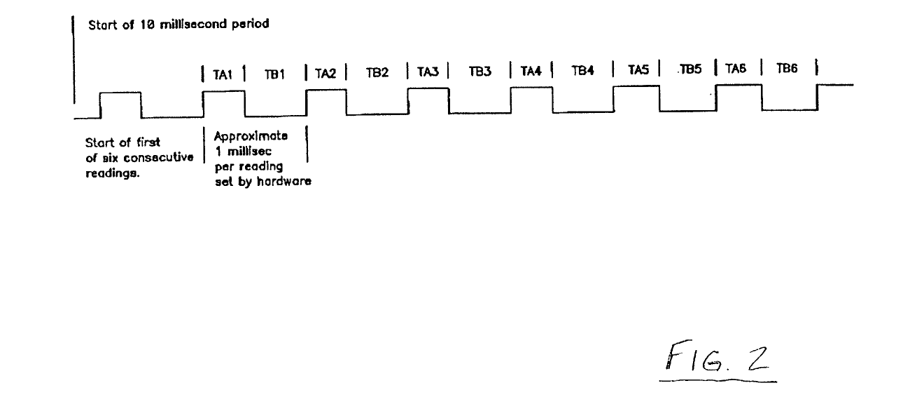

FIG. 2 is diagram illustrating a square wave signal produced by an accelerometer;

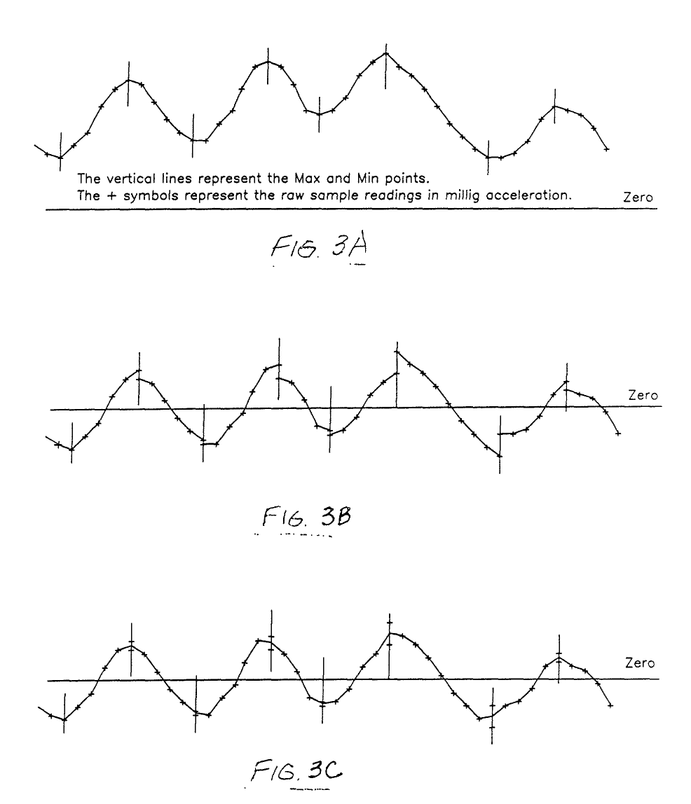

FIG. 3A is a diagram illustrating an oscillating signal comprised of the ten millisecond samples derived from the square wave signal of FIG. 2;

FIG. 3B is a diagram illustrating adjustment of the signal of FIG. 3A to generally be centered about the horizontal axis of FIG. 3A;

FIG. 3C is a diagram further illustrating adjustment of the signal of FIG. 3A about the horizontal axis of FIG. 3A;

FIG. 4A is a diagram illustrating an oscillating signal comprised of ten millisecond samples derived from a square wave signal comparable to that of FIG. 2 along the F/B axis of the accelerometer of FIG. 1 while a patient moves his right arm;

FIG. 4B is a diagram illustrating the oscillating signal of FIG. 4A after each point is averaged with the next two consecutive points;

FIG. 4C is a diagram illustrating the oscillating signal of FIG. 4B after it has been adjusted with a min-max averaging to generally center the signal around the horizontal (zero) acceleration axis;

FIG. 4D is a diagram illustrating the oscillating signal of FIG. 4C after it has been subjected to a zero offset adjustment by adding to each point a positive value equal in magnitude to the lowest negative value of the signal of FIG. 4C;

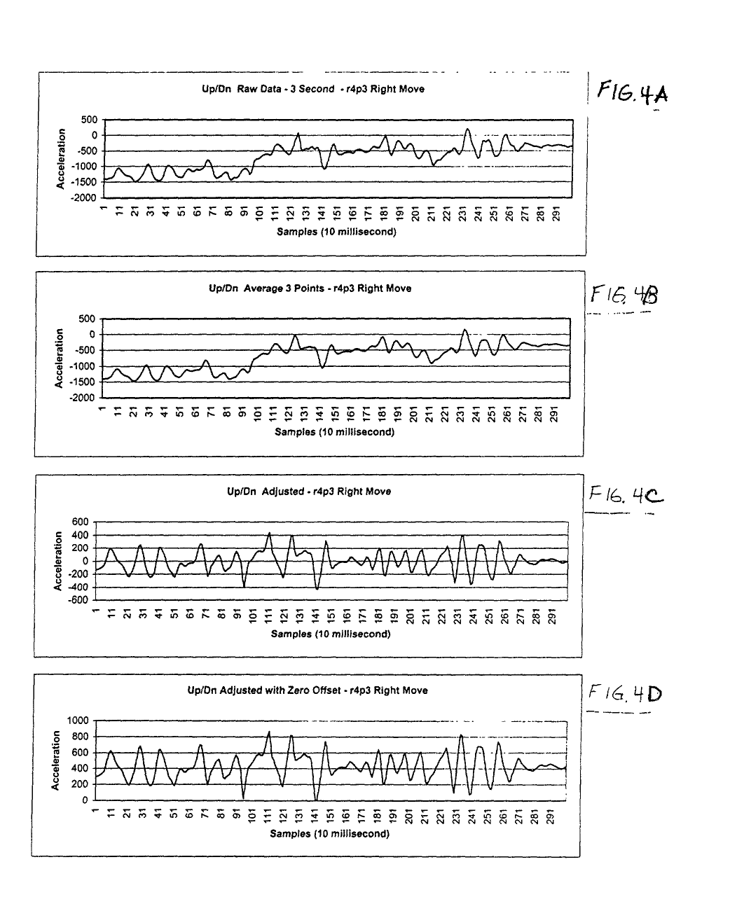

FIG. 5A is a diagram illustrating an oscillating signal comprised of ten millisecond samples derived from a square wave signal comparable to that of FIG. 2 along the R/L axis of the accelerometer of FIG. 1 while a patient moves his right arm;

FIG. 5B is a diagram illustrating the oscillating signal of FIG. 5A after each point is averaged with the next two consecutive points;

FIG. 5C is a diagram illustrating the oscillating signal of FIG. 5B after it has been adjusted with a min-max averaging to generally center the signal around the horizontal (zero) acceleration axis;

FIG. 5D is a diagram illustrating the oscillating signal of FIG. 5C after it has been subjected to a zero offset adjustment by adding to each point a positive value equal in magnitude to the lowest negative value of the signal of FIG. 5C;

FIG. 6A is a diagram illustrating an oscillating signal comprised of ten millisecond samples derived from a square wave signal comparable to that of FIG. 2 along the U/D axis of the accelerometer of FIG. 1 while a patient moves his right arm;

FIG. 6B is a diagram illustrating the oscillating signal of FIG. 6A after each point is averaged with the next two consecutive points;

FIG. 6C is a diagram illustrating the oscillating signal of FIG. 6B after it has been adjusted with a min-max averaging to generally center the signal around the horizontal (zero) acceleration axis;

FIG. 6D is a diagram illustrating the oscillating signal of FIG. 6C after it has been subjected to a zero offset adjustment by adding to each point a positive value equal in magnitude to the lowest negative value of the signal of FIG. 6C;

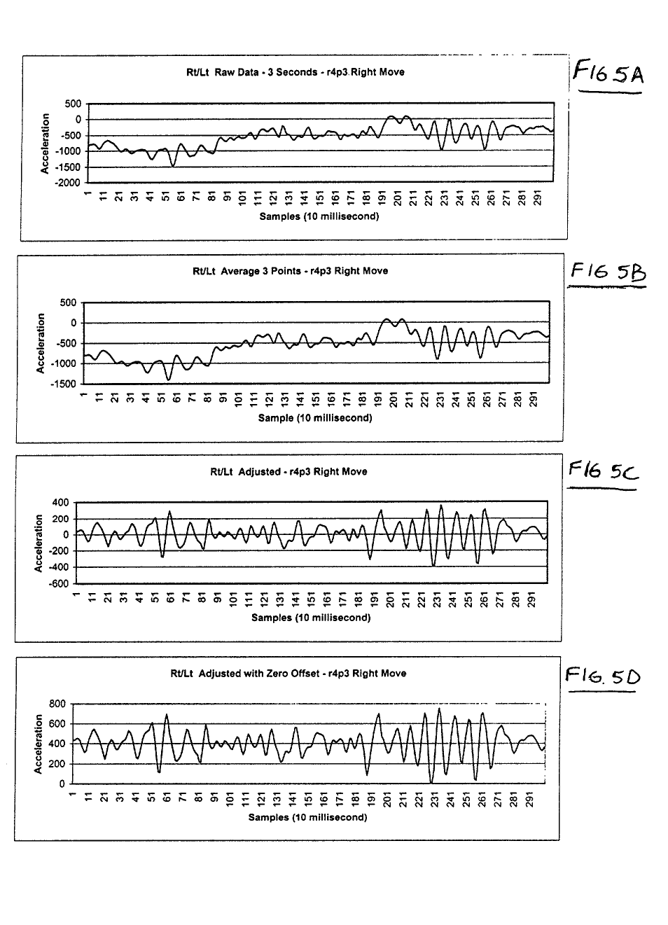

FIG. 7A is a diagram illustrating the oscillating signal produced by, for each sample comprising the signals of FIGS. 4D, 5D, 6D, squaring the value of the sample and adding together the resulting three values;

FIG. 7B is a diagram illustrating the composite oscillating signal produced by taking the square root of the value of each of the samples of FIG. 7A;

FIG. 7C is a diagram illustrating the composite oscillating signal of FIG. 7B adjusted with min-max averaging to generally center the signal about the zero axis;

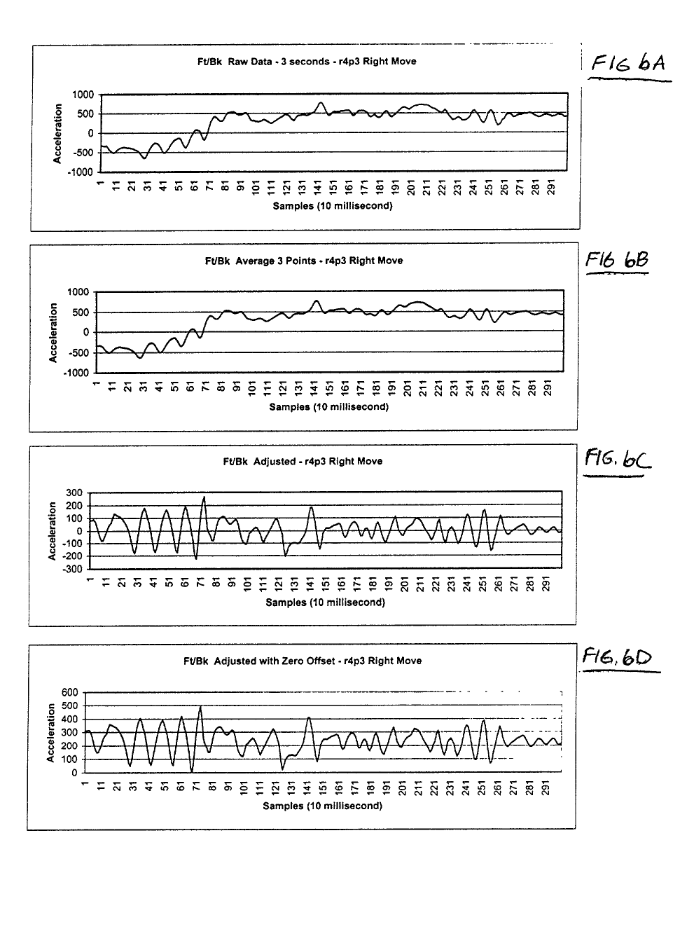

FIG. 8 is a diagram illustrating the composite oscillating signal of FIG. 7C along with the remainder of the samples taken during about a twenty-one second period of time;

FIG. 9A is a diagram illustrating the signal of FIG. 6A along with the remainder of the samples taken during about a twenty-one second period of time;

FIG. 9B is a diagram illustrating the signal of FIG. 5A along with the remainder of the samples taken during about a twenty-one second period of time;

FIG. 9C is a diagram illustrating the signal of FIG. 4A along with the remainder of the samples taken during about a twenty-one second period of time;

FIG. 9D is a diagram of a composite signal identical to that of FIG. 8;

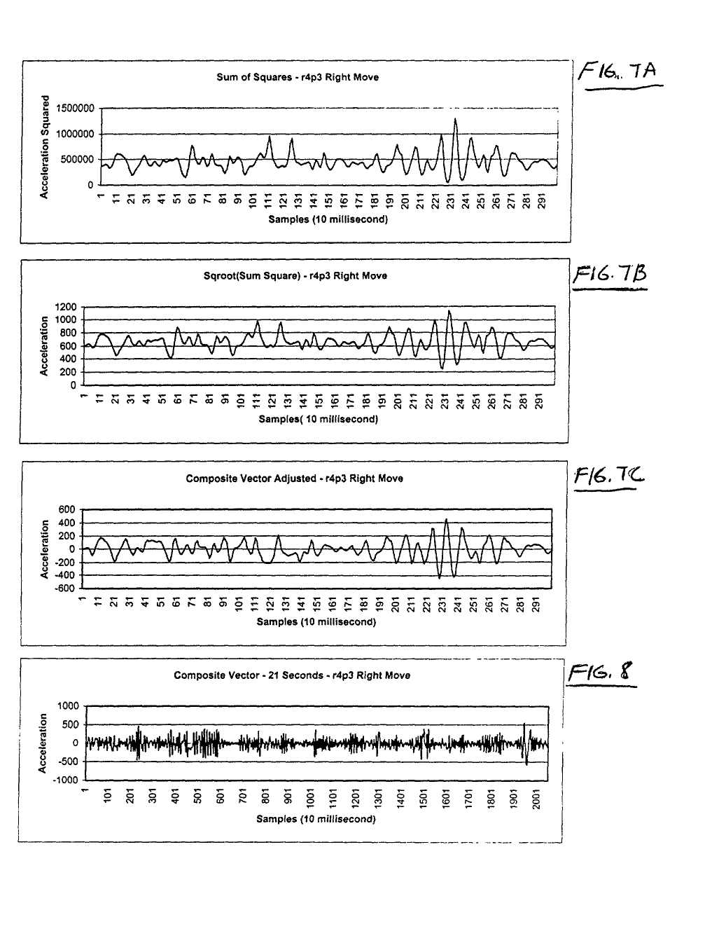

FIG. 10 is a diagram of a composite signal generated in the same manner as the diagram of FIG. 8, but for when a patient holds his right arm away from his body and parallel to the ground;

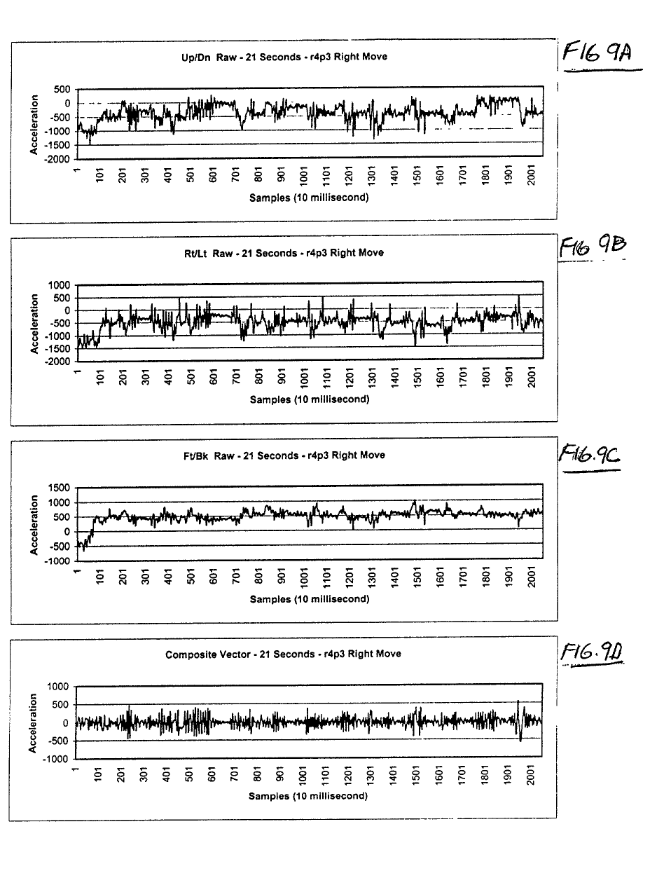

FIG. 11 is a diagram illustrating the analysis of one cycle in the composite signal of FIG. 10;

FIG. 12 is a diagram generated from frequency data produced during analysis of the cycles in the signal of FIG. 10;

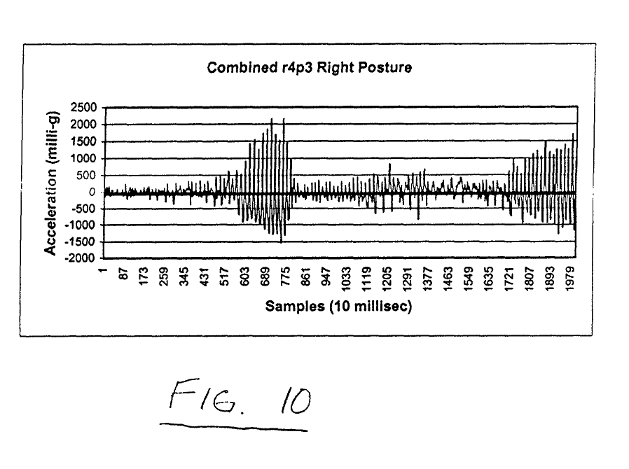

FIG. 13 is a diagram generated from area data produced during analysis of the cycles in the signal of FIG. 11;

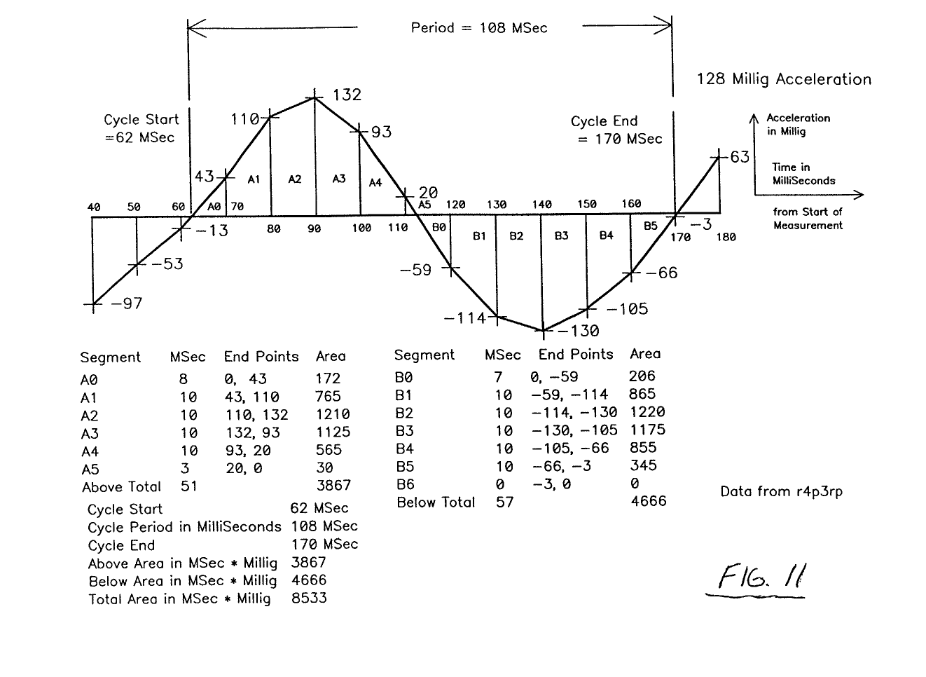

FIG. 14 is a diagram generated from amplitude data produced during analysis of the cycles in the signal of FIG. 11;

FIG. 15 is a diagram generated from frequency data produced during analysis of the cycles in the signal of FIG. 11;

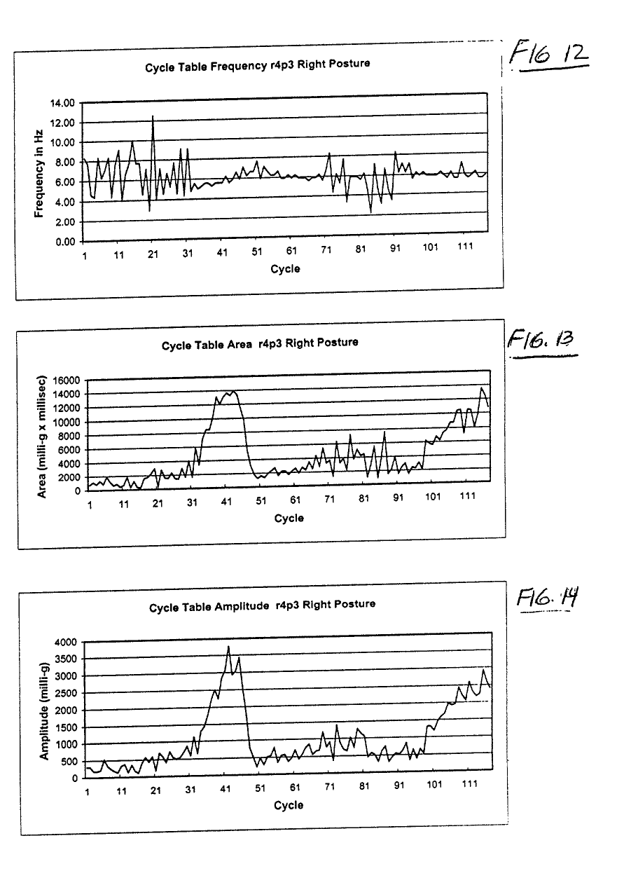

FIG. 16 is a diagram illustrating generation of a tremor selection line based on cycle-frequency data produced during analysis of the cycles in the signal of FIG. 11;

FIG. 17 is a diagram illustrating generation of a tremor selection line based on cycle-area data produced during analysis of the cycles in the signal of FIG. 11;

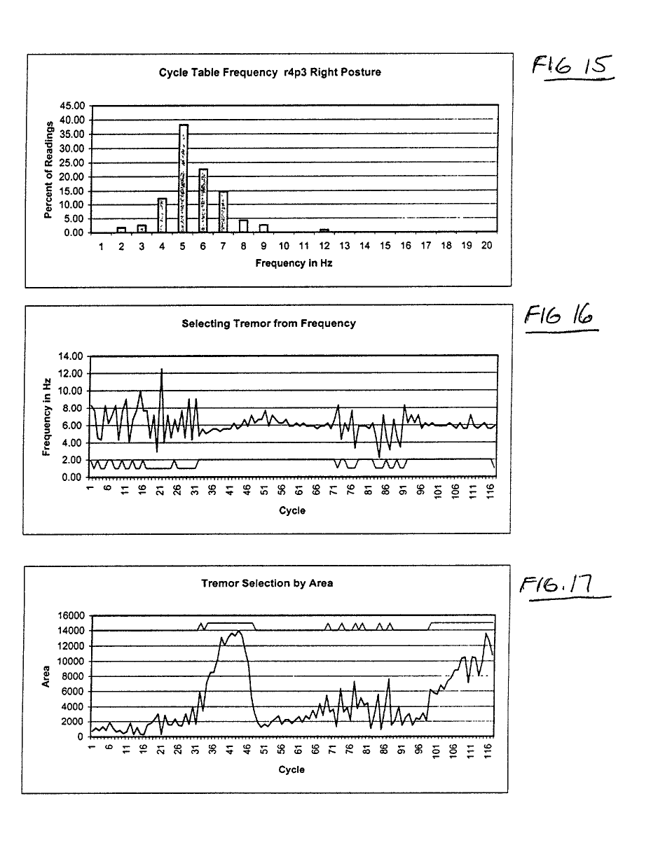

FIG. 18 is a diagram produced by multiplying together the tremor selection line of FIG. 16 and the tremor selection line of FIG. 17;

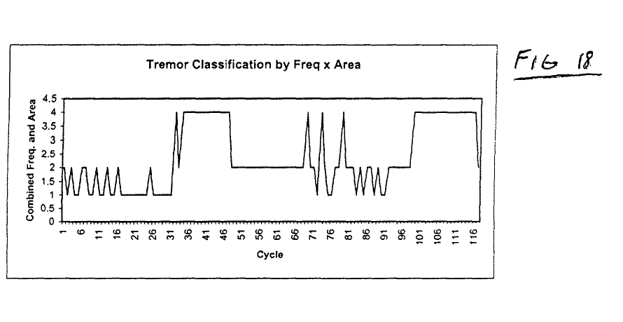

FIG. 19 is a diagram illustrating the mean and standard deviation of frequency data produced during analysis of the cycles in the signal of FIG. 11 with respect to posture and of data produced during analysis of cycles in signals produced for rest, load, and move by using a procedure identical to that used to produce the signal of FIG. 11;

FIG. 20 is a frequency percent histogram illustrating data produced by analyzing cycles in signals produced during rest, posture, load, and move;

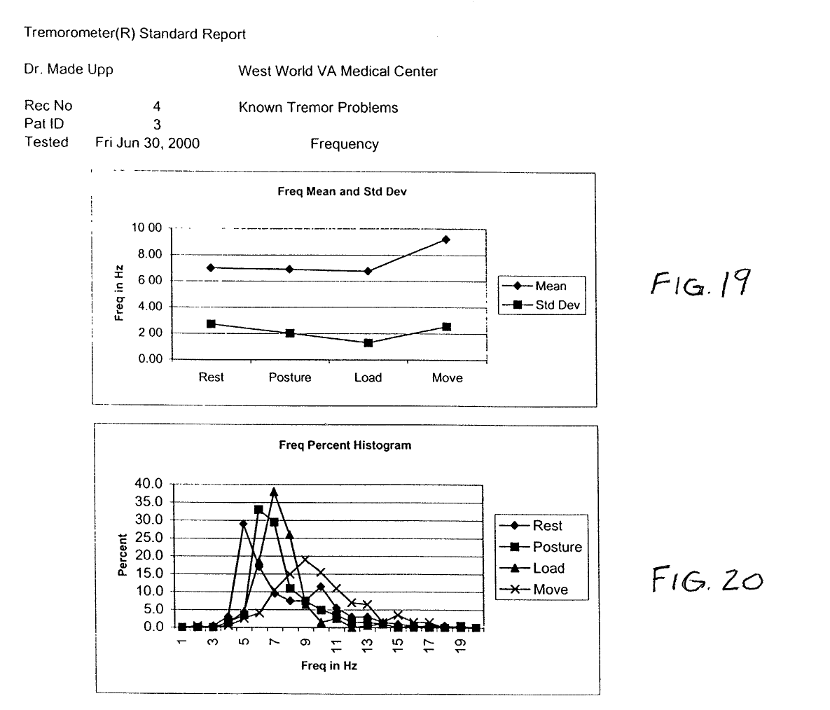

FIG. 21 is a diagram illustrating the mean and standard deviation of amplitude data produced during analysis of the cycles in the signal of FIG. 11 with respect to posture and of data produced during analysis of cycles in signals produced for rest, load, and move by using a procedure identical to that used to produce the signal of FIG. 11;

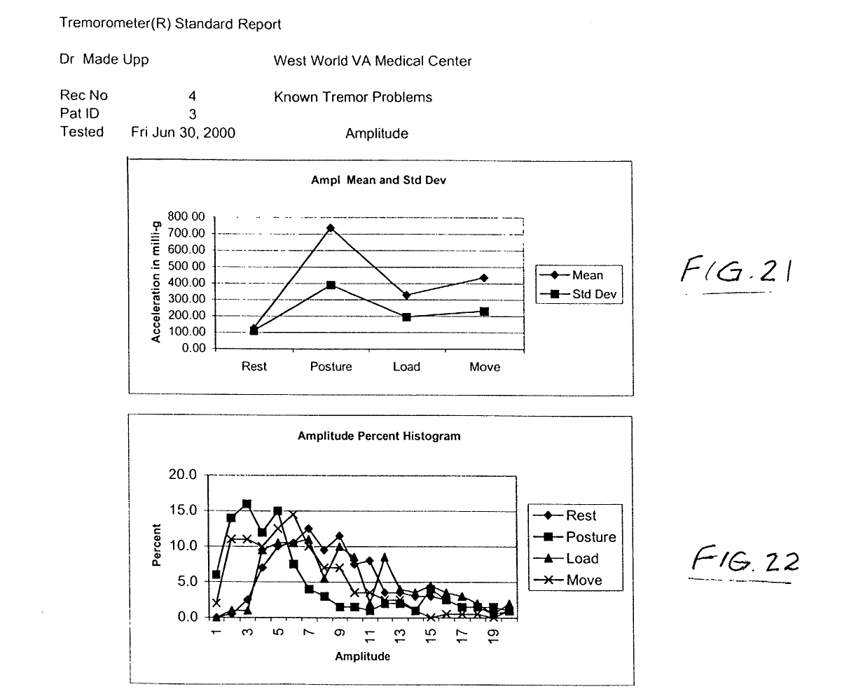

FIG. 22 is an amplitude percent histogram illustrating data produced by analyzing cycles in signals produced during rest, posture, load, and move;

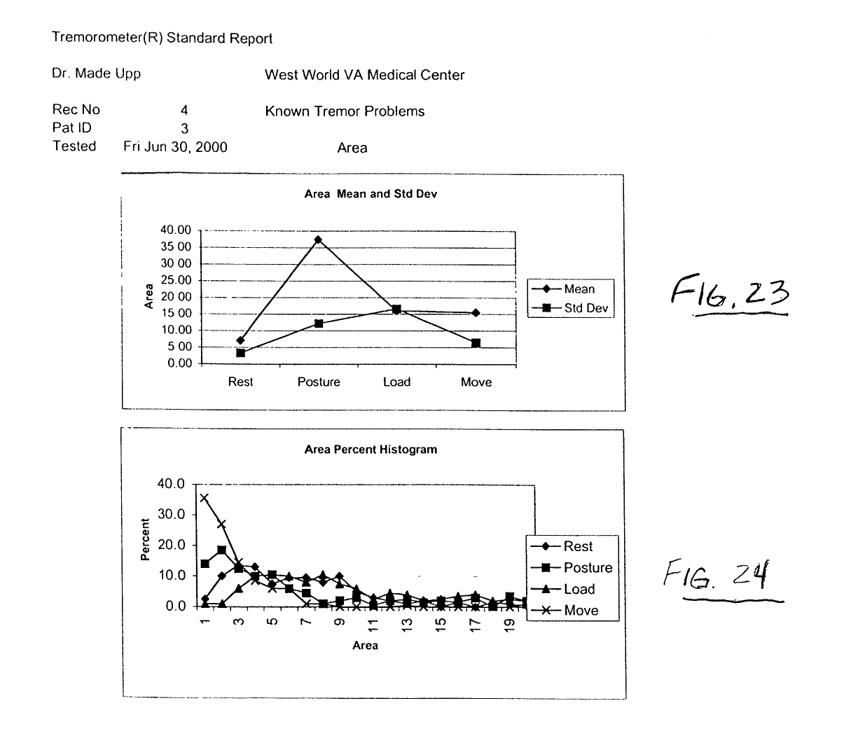

FIG. 23 is a diagram illustrating the mean and standard deviation of area data produced during analysis of the cycles in the signal of FIG. 11 with respect to posture and of data produced during analysis of cycles in signals produced for rest, load, and move by using a procedure identical to that used to produce the signal of FIG. 11;

FIG. 24 is an area percent histogram illustrating data produced by analyzing cycles in signals produced during rest, posture, load, and move;

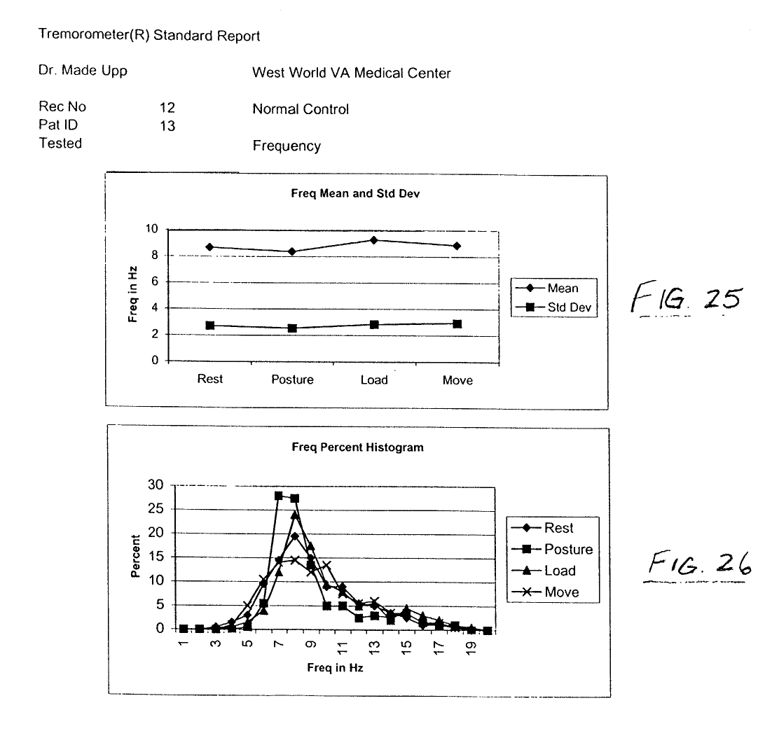

FIG. 25 is a diagram illustrating the mean and standard deviation of frequency data produced during analysis of the cycles in signals produced for posture, rest, load, and move by using a procedure identical to that used to produce the signal of FIG. 11;

FIG. 26 is a frequency percent histogram illustrating data produced by analyzing cycles in signals produced during rest, posture, load, and move;

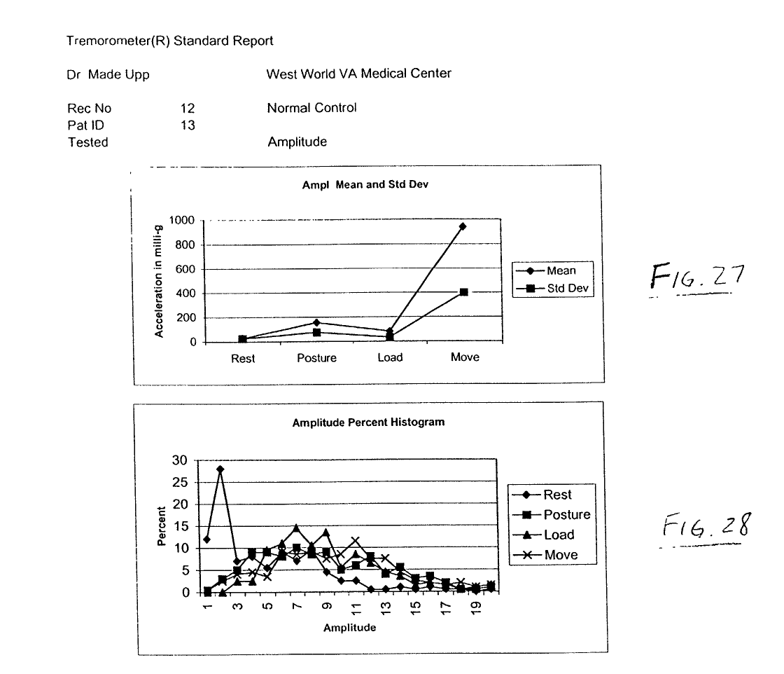

FIG. 27 is a diagram illustrating the mean and standard deviation of amplitude data produced during analysis of the cycles in signals produced for posture, rest, load, and move by using a procedure identical to that used to produce the signal of FIG. 11;

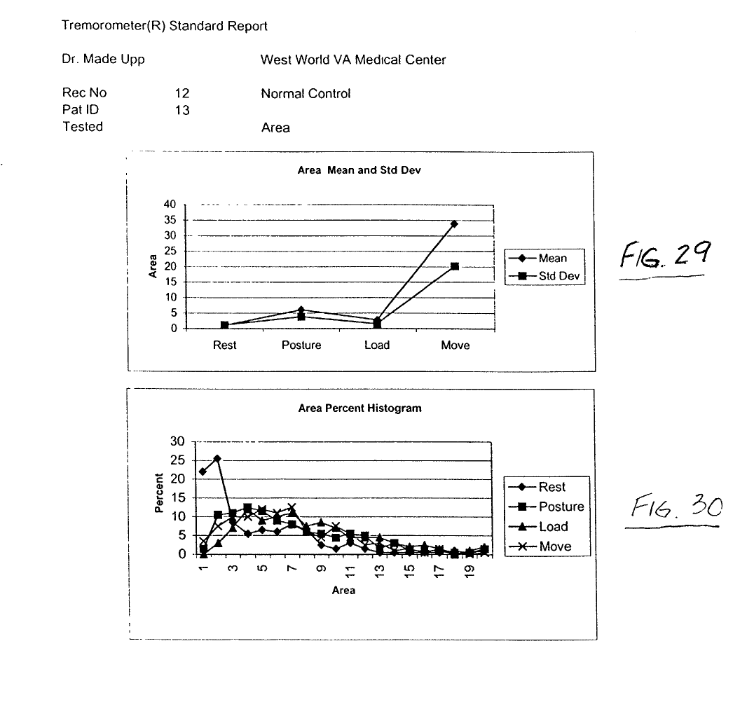

FIG. 28 is an amplitude percent histogram illustrating data produced by analyzing cycles in signals produced during rest, posture, load, and move;

FIG. 29 is a diagram illustrating the mean and standard deviation of area data produced during analysis of the cycles in signals produced for posture, rest, load, and move by using a procedure identical to that used to produce the signal of FIG. 11;

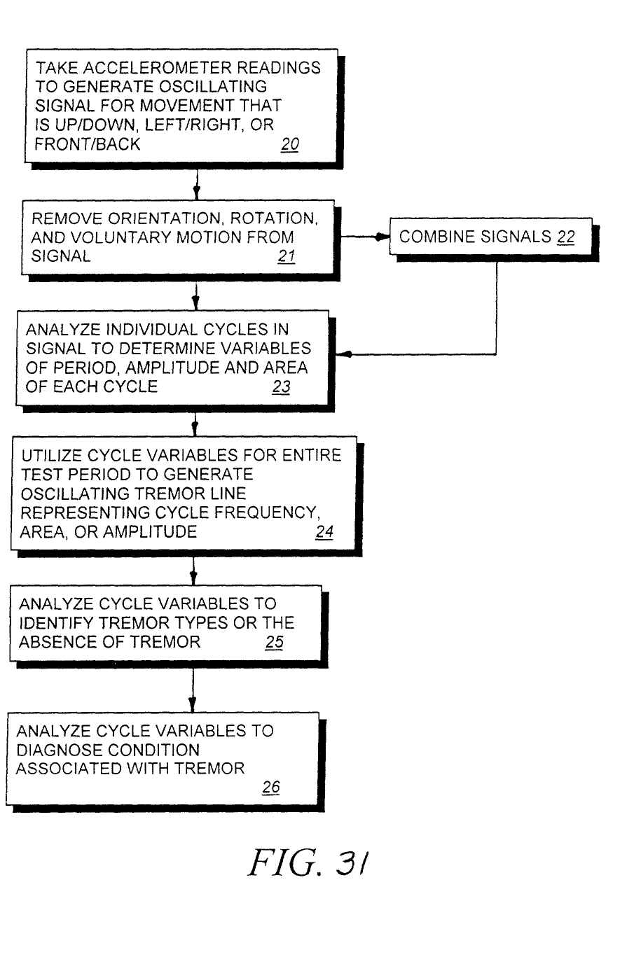

FIG. 30 is an area percent histogram illustrating data produced by analyzing cycles in signals produced during rest, posture, load, and move; and, FIG. 31 is a block flow diagram illustrating a methodology of implementing the invention.

Briefly, in accordance with the invention, I provide an improved apparatus for generating a filtered tremor signal representing tremor in a portion of the body of a patient. The apparatus includes measurement apparatus for measuring tremor to generate a raw signal comprising a plurality of samples each indicating acceleration; and, apparatus for filtering the raw signal to eliminate at least a portion of high frequency noise in the raw signal, orientation, rotation, and voluntary motion.

In another embodiment of the invention, I provide improved apparatus for generating data representing tremor in a portion of the body of a patient. The apparatus includes measurement apparatus for measuring tremor to generate a cyclical signal comprising a plurality of samples each indicating acceleration, the signal oscillating about a selected reference line, and, for examining the signal to define for each cycle comprising the cyclical signal the beginning point of the cycle, the ending point of the cycle, the maximum amplitude of the cycle, the minimum amplitude of the cycle, the area of the cycle above the reference line, and the area of the cycle below the reference line.

In a further embodiment of the invention, I provide improved apparatus for generating data representing tremor in a portion of the body of a patient. The apparatus includes measurement apparatus for measuring tremor to generate a cyclical signal comprising a plurality of samples each indicating acceleration, said signal oscillating about a selected reference line; and, apparatus for generating data indicating the frequency of each cycle in the cyclical signal.

In still another embodiment of the invention, I provide improved apparatus for generating data representing tremor in a portion of the body of a patient. The improved apparatus includes measurement apparatus for measuring tremor to generate a cyclical signal comprising a plurality of samples each indicating acceleration, the signal oscillating about a selected reference line; and, apparatus for generating data indicating the area of each cycle in said cyclical signal.

In yet a further embodiment of the invention, I provide improved apparatus for generating data representing tremor in a portion of the body of a patient. The apparatus includes measurement apparatus for measuring tremor to generate a cyclical signal comprising a plurality of samples each indicating acceleration, the signal oscillating about a selected reference line; and, apparatus for generating data indicating the amplitude of each cycle in the cyclical signal.

In yet still another embodiment of the invention, I provide improved apparatus for generating data representing tremor in a portion of the body of a patient. The apparatus includes measurement apparatus for measuring tremor to generate a cyclical signal comprising a plurality of samples each indicating acceleration, the signal oscillating about a selected reference line; and, apparatus for generating data for a selected grouping of consecutive cycles indicating at least one of a group comprising the area of each cycle in the grouping of consecutive cycles, the frequency of each cycle in the grouping of consecutive cycles, and the amplitude of each cycle in the grouping of consecutive cycles.

In a further embodiment of the invention, I provide improved apparatus for identifying a condition associated with a patient's tremor. The apparatus includes measurement apparatus for measuring tremor to generate a cyclical signal comprising a plurality of samples each indicating acceleration; apparatus for generating data indicating at least one characteristic of the cycles comprising the cyclical signals; apparatus for generating a database indicating values of the characteristic for a particular condition; and, apparatus correlating said data with said database to determine the likelihood of the patient's tremor being associated with said condition.

In another embodiment of the invention, I provide an improved method for measuring tremor and identifying a condition associated with a patient's tremor. The method includes the steps of measuring tremor to generate a cyclical signal comprising a plurality of samples each indicating acceleration; generating data indicating at least one characteristic of the cycles comprising the cyclical signals; generating a database indicating values of the characteristic for a particular condition; and, correlating the data with the database to determine the likelihood of the patient's tremor being associated with the condition.

Turning now to the drawings, which depict the presently preferred embodiments of the invention for the purpose of illustrating the practice thereof and not byway of limitation of the scope of the invention, and in which like reference characters refer to corresponding elements throughout the several views, FIG. 1 illustrates an accelerometer 10. The accelerometer produces signals for movement along the U/D (up/down) axis 11, F/B (front/back) axis 12, R/L (right/left) axis 13.

FIG. 2 illustrates a sample measurement “square wave” pulse width modulated direct current (DC) signal produced by accelerometer 10 for one of the axes 11, 12, 13. The measurement signal produced by accelerometer 10 for each axis 11, 12, 13 is a square wave DC signal of the type illustrated in FIG. 2. The DC signal of FIG. 2 is utilized by taking during each ten millisecond sampling period six readings. The number of readings taken during the ten millisecond period can vary as desired, as can the length of the sampling period. In FIG. 2, the length of time consumed by each reading pair TA1-TB1, TA2-TB2, etc. is about one millisecond.

The signal of FIG. 2 is received by a microprocessor which determines the amount of time that the DC signal is high by adding together the time span for each high signal TA1, TA2, TA3, etc.:

TA SUM=TA1+TA2+ . . . +TA6

The microprocessor also determines the amount of time that the DC signal is low by adding together the time span for each low signal TB1, TB2, etc.:

TB SUM=TB1+TB2+TB3+ . . . +TB6

The relative acceleration (REL ACC) is calculated by dividing the total time span TA SUM for the high readings by the total time span for both the high and low readings:

REL ACC=TA SUM/(TA SUM+TB SUM)

Absolute acceleration (ABS ACC) is calculated by multiplying the relative acceleration by a calibration constant (CAL CON) and adding a calibration zero offset (CAL ZERO):

ABS ACC=(REL ACC×CAL CON)+CAL ZERO

The calibration constant and the calibration zero offset are generated by a procedure that uses the earth's gravity acceleration as a reference. The foregoing procedure produces an absolute acceleration sample every ten milliseconds for each axis 11, 12, 13 of the accelerometer 10. Each absolute acceleration sample is called a raw reading.

The raw readings include signal components generated when the individual rotates his body part and also includes signal components generated due to the orientation of the accelerometer to earth's gravity. By way of example, rotation of a patient's arm occurs when the arm is turned in a direction of travel which circumscribes the longitudinal axis of the arm. For example, if the elbow and upper arm are basically stationary and horizontal and a patient's palm is facing up, when the patient turns his hand so the palm faces down, the forearm is rotated about the longitudinal axis of the forearm.

Since it is more efficient in time and computer code to use integer arithmetic than to use floating point arithmetic, all of the computer routines preferably are written in integers. A special divide function is used to produce rounding that follows the IEEE 754 specification. The call

C=divide(A,B)

will return the correct result in C from the operation A/B with the proper rounding such that the fractional parts of division are handled correctly and with a remainder of exactly 0.50 being rounded to the nearest even whole number.

As is illustrated in FIG. 3A, the raw readings are not centered on the horizontal axis 14, typically because of the rotation and orientation components noted above. To compensate for and remove the rotation and orientation components from the raw readings, the Min (minimum) and Max (maximum) points are located for each cycle segment and are averaged:

MIN/MAX VALUE=divide((Max+Min),2).

This MIN/MAX VALUE is subtracted from each raw reading, producing the signal illustrated in FIG. 3B. In order to compensate for the offset between cycle segments in FIG. 3B, the high point of a cycle segment is averaged with the high point of the next adjacent cycle segment, and, the low point of a cycle segment is averaged with the low point of the next adjacent cycle segment. This averaging produces the signal illustrated in FIG. 3C.

The procedure described in connection with FIGS. 3A to 3C is utilized in connection with FIGS. 4A to 4C. FIG. 4A illustrates the raw data (samples) obtained from an accelerometer for the U/D axis during the first few seconds of “Right Move”. As used herein, “Right Move” means that the patient begins with his right arm extended horizontally out from his body and then moves continuously his hand between his nose and the horizontally extended position during the entire twenty one second test period. “Right Rest” means the patient maintains his right arm in a vertically oriented position at his side during the entire twenty-one second test period. “Right Posture” means the patient maintains his right arm in a horizontal position extending outwardly from his body. “Right Load” means that during the entire twenty-one second test period the patient maintains his right arm in a horizontal position extending outwardly from his body while holding a weight in his hand. “Move” means the patient is moving a body part while accelerometer measurements are being taken. “Rest” means the body part is at rest while accelerometer measurements are being taken. “Posture” means the body part is held in a fixed position requiring exertion on the part of the patient to maintain the body part in a fixed position. “Load” means the body part is undergoing exertion and supporting a weight while accelerometer measurements are being taken.

FIG. 4B represents the signal of FIG. 4A after each three consecutive samples are averaged. For example, the acceleration values for points 1, 2, 3 in FIG. 4A are added and divided by three and the average is used to replace the value for point 2. Then the acceleration values for points 2, 3, 4 are added and divided by three and the average is used to replace the value for point 3. And so on.

FIG. 4C represents the signal of FIG. 4B after the signal of 4B is subjected to the MIN/MAX adjustment described with respect to and illustrated in FIGS. 3B and 3C.

FIG. 4D represents the signal of 4C after a positive number equal in magnitude to the greatest negative acceleration sample in FIG. 4C (i.e., the negative acceleration near sample 141) is added to the value of each sample in FIG. 4C.

The procedure described in connection with FIGS. 3A to 3C is utilized in connection with FIGS. 5A to 5C. FIG. 5A illustrates the raw data (samples) obtained from an accelerometer for the R/L axis during the first few seconds of “Right Move”.

FIG. 5B represents the signal of FIG. 5A after each three consecutive samples are averaged. For example, the acceleration values for points 1, 2, 3 in FIG. 5A are added and divided by three and the average is used to replace the value for point 2. Then the acceleration values for points 2, 3, 4 are added and divided by three and the average is used to replace the value for point 3. And so on.

FIG. 5C represents the signal of FIG. 5B after the signal of 5B is subjected to the MIN/MAX adjustment described with respect to and illustrated in FIGS. 3B and 3C.

FIG. 5D represents the signal of 5C after a positive number equal in magnitude to the greatest negative acceleration sample in FIG. 5C (i.e., the negative acceleration near sample 231) is added to the value of each sample in FIG. 5C.

The procedure described in connection with FIGS. 3A to 3C is utilized in connection with FIGS. 6A to 6C. FIG. 6A illustrates the raw data (samples) obtained from an accelerometer for the F/B axis during the first few seconds of “Right Move”.

FIG. 6B represents the signal of FIG. 6A after each three consecutive samples are averaged. For example, the acceleration values for points 1, 2, 3 in FIG. 6A are added and divided by three and the average is used to replace the value for point 2. Then the acceleration values for points 2, 3, 4 are added and divided by three and the average is used to replace the value for point 3. And so on.

FIG. 6C represents the signal of FIG. 6B after the signal of 6B is subjected to the MIN/MAX adjustment described with respect to and illustrated in FIGS. 3B and 3C.

FIG. 6D represents the signal of 6C after a positive number equal in magnitude to the greatest negative acceleration sample in FIG. 6C (i.e., the negative acceleration near sample 71) is added to the value of each sample in FIG. 6C.

FIG. 7A is a diagram illustrating the signal produced by, for each sample comprising the signals of FIGS. 4D, 5D, 6D, squaring the value of the sample and adding together the resulting three values. For example, the acceleration value for sample 1 of FIG. 4D is squared; the acceleration value for sample 1 of FIG. 5D is squared; and, the acceleration value for sample 1 of FIG. 6D is squared. These three squared values are added together to produce the value shown in FIG. 7A.

FIG. 7B is a diagram illustrating the composite oscillating signal produced by taking the square root of each of the sample values of FIG. 7A. For example, the square root of the acceleration value for sample 1 in FIG. 7A is the acceleration value plotted in FIG. 7B for sample 1.

FIG. 7C is a diagram illustrating the composite oscillating signal of FIG. 7B adjusted by the min-max averaging procedure discussed above with respect to FIGS. 3B and 3C. The min-max averaging procedure functions to generally center the composite signal of FIG. 7B around the zero acceleration axis of FIG. 7C.

FIG. 8 is a diagram illustrating the composite oscillating signal of FIG. 7C along with the remainder of the samples obtained from accelerometer signals during about a twenty-one second period of time that accelerometer measurements are being recorded.

FIG. 9A is a diagram illustrating the signal of FIG. 4A along with the remainder of the raw samples obtained from accelerometer signals during about a twenty-one second period of time.

FIG. 9B is a diagram illustrating the signal of FIG. 5A along with the remainder of the samples obtained from accelerometer signals during about a twenty-one second period of time.

FIG. 9C is a diagram illustrating the signal of FIG. 6A along with the remainder of the samples obtained from accelerometer signals during about a twenty-one second period of time.

FIG. 9D is a diagram of a composite signal and is identical to the diagram of FIG. 8.

FIG. 10 is a diagram of a composite signal produced utilizing the same procedure utilized to produce the composite signal in the diagram of FIG. 8. However, the composite signal in FIG. 10 is generated for right posture. Consequently, while a patient holds his right arm out for a period of about twenty-one seconds, an accelerometer 10 mounted on the patient's hand generates signals for the U/D, R/L, F/B axes 11, 12, 13, of the accelerometer 10. These signals are processed in the manner earlier described with reference to FIGS. 2 to 9 to produce the composite signal of FIG. 10 from the right posture accelerometer signals.

FIG. 11 is a diagram of one cycle from the composite signal of FIG. 10. This cycle is analyzed in the manner described below to produce data defining the cycle. Each cycle in the composite signal of FIG. 10 is analyzed in the same manner.

Since the signal of FIG. 10 can generally be characterized as a sine wave, the first step in analyzing the cycle of FIG. 11 is to determine the “zero crossing” points defining the beginning, midpoint, and end of the cycle. It is reasonable to use a linear interpolation to find the zero crossing based on the line connecting the points on either side of each zero crossing.

The algorithms for finding the zero crossing to the nearest millisecond are quite simple. Assuming that the line connecting the point below (for example point −13 at sixty milliseconds in FIG. 11) and the point above (for example point +43 at seventy milliseconds in FIG. 11) the horizontal line or axis in FIG. 11 is a straight line, two similar triangles are formed which have the following relationship.

AX/AY=BX/BY

AX and BX are segments along the X axis (horizontal axis in FIG. 11) representing time which are unknown. AY and BY are measurements of acceleration above and below the X axis and the point of zero crossing. A point of zero crossing is the point at which the sine wave crosses the X axis.

We know that:

AX+BX=one sample period=ten milliseconds

Then:

AX=AY/(AY−BY)

This generates the fraction of the total sample period that occurs while the sine wave is above the zero crossing.

Then:

AX×10 equals the time above in milliseconds

And:

BX=10−milliseconds,

is a simple way to obtain the portion below the zero crossing.

All that is required to convert the ten millisecond readings into fairly accurate millisecond values is to find the above and below readings at the zero crossing, to perform the simple math, and to add the millisecond portion to the ten millisecond sample to form a single millisecond term:

(Sample×10)+milliseconds,

to add the segment of the cycle above the zero crossing.

Or:

(Sample×10)+(10−milliseconds),

for the segment below the zero crossing.

Milliseconds=divide((AY×10),(AY−BY)),

where the 10 is the conversion from ten millisecond sample to milliseconds. The BY value is by definition negative, being below the zero crossing so that the (AY−BY) calculation actually adds the absolute value of the amplitude above and below the line.

If desired, a calculation procedure other than that just described can be used to estimate and assign values to the zero crossing points for a cycle. Any calculation procedure utilized is preferably accurate to within plus or minus 2%. While it is not necessary to assume that the line between two consecutive points (for example points 13 and +43 in FIG. 11), one above and one below the zero axis, is straight, such an assumption simplifies calculations and is believed to produce a reasonable estimate of the zero crossing point.

The area “under the curves” (i.e., between the cycle and the horizontal zero axis) is calculated. In FIG. 11, the cycle is divided into segments each having a width equal to ten milliseconds. If a segment does not include a zero crossing point, the area is:

Area of Segment=divide(Abs(AY+BY),2)×10,

where “Abs” indicates that the absolute value is being taken to handle number pairs which are below zero and have a negative value, where (AY+BY) is the sum of the two readings from the portion, where the division by two is to find the average of the two readings, where the multiplication by ten is to convert from ten millisecond samples to millisecond values.

Area of Segment=Abs(AY+BY)×5,

is a simplified version of the above “Area of Segment” formula, and since it does not use any division, it does not have a rounding problem.

When the segment has a zero crossing, the segment of the signal above the zero crossing must be handled separately from the segment of the signal below the zero crossing.

Area of Segment Above=divide((AY×milliseconds),2),

where AY is multiplied by the number of milliseconds the signal is above and the value is divided by two since the area between the zero crossing and the AY value is a triangle.

Area of Segment Below=divide((−BY×(10−milliseconds)),2),

which is similar to the calculation for the Area of the Segment Above. However, the minus at the beginning of the calculation for Area of Segment Below is required to convert the negative value into a positive area value and the “10−milliseconds” calculation is required to convert the time from the positive portion of the zero crossing to the negative portion.

The start of a cycle is presently defined as a positive zero crossing, and the area of the segment above the horizontal zero or X axis is calculated and is used as the total area initialization.

Each segment without a zero crossing is calculated and added to the total area.

When the cycle crosses the horizontal zero axis and goes from positive to negative, both the area above segment and the area below segment are separately calculated and each is added to the total area of the cycle.

The end of the cycle is defined as the next positive zero crossing, i.e., where the signal line crosses the horizontal zero axis and goes from negative to positive. At the end of the cycle, the segment below portion is calculated and added to the total area as the last component of the total area.

In the practice of the invention, the foregoing procedure for calculating the area between the cycle and the horizontal zero axis need not be utilized. Any other desired procedure for calculating the area can be utilized. Any calculation procedure utilized preferably, but not necessarily, is accurate to within plus or minus 2% of the actual area.

FIG. 11 depicts the area of each segment of the cycle which begins with segment A0 (which is above the horizontal X axis) and ends with segment B5 (which is below the horizontal X axis), depicts the total area of 8533 in msec×millig, depicts the start of the cycle at 62 msec, depicts the end of the cycle at 170 msec, depicts the period of the cycle at 108 msec, depicts the maximum value or amplitude of the cycle at 132 millig acceleration, and depicts the minimum value or amplitude of the cycle at −130 millig. As noted, such values are calculated for each cycle in the composition signal illustrated in FIG. 10 for right posture and are calculated for each cycle in composite signals produced for right load, right rest, and right move, or, for left rest, left posture, left move, and left load, or, for composite signals produced based on accelerometer readings for other portions of the body. The cycle analysis illustrated above can also, if desired, be carried out on the signals of FIGS. 4C, 5C, 6C, or on any of the other signals illustrated in FIGS. 4 to 9. It is presently preferred, however, to utilize composite signals of the type set forth in FIG. 10.

Tables I and II on the following pages list by way of example, data calculated for the initial forty-one cycles in the composite signal of FIG. 10. Tables I to XIV are grouped at the end of this specification.

FIG. 12 utilizes data from Tables I and 11 and is a graphical representation of the frequency in Hz of each of the cycles in the composite signal of FIG. 10.

FIG. 13 utilizes data from Tables I and 11 and is a graphical representation of the area in millig×millisecond of each of the cycles in the composite signal of FIG. 10.

FIG. 14 utilizes data from Tables I and 11 and is a graphical representation of the amplitude in millig of each of the cycles in the composite signal of FIG. 10.

FIG. 15 utilizes data from Tables I and II and illustrates how often particular frequencies occur.

FIG. 16 illustrates the frequency vs. cycle graph of FIG. 12 and also illustrates a tremor selection line which extends horizontally across the graph between one and two Hz. The tremor selection line is a logic level that is set to low each time a cycle has a frequency which is two or more Hz greater than the frequency of the previous cycle in the frequency vs cycle graph of FIG. 12, and is set to high each time a cycle has a frequency less than two Hz greater than the frequency of the previous cycle in the frequency vs cycle graph of FIG. 12.

FIG. 17 illustrates the area vs. cycle graph of FIG. 13 and also illustrates a tremor selection line which extends horizontally across the graph between 14,000 and 15,000. The tremor selection line of FIG. 17 is a logic level that is set to low when the area of a cycle is equal to or greater than the mean area of the cycles in the area vs. cycle graph of FIG. 13, and is set to high when the area of a cycle is less than the mean area of the cycles in the area vs. cycle graph of FIG. 13.

FIG. 18 is an illustration of a graph produced by multiplying the value of cycle “1” in the tremor frequency selection line of FIG. 16 by the value of cycle “1” in the tremor area selection line of FIG. 17; by multiplying the value of cycle “2” in the tremor frequency selection line of FIG. 16 by the value of cycle “2” in the tremor area selection line of FIG. 17, etc.

Table III below sets forth the mean, standard deviation, and median calculations for the frequencies set forth in frequency vs. cycle graph of FIG. 12 (right posture, “R Posture”), as well as for additional frequency vs. cycle graphs that were generated (but not shown here) for right rest (“R Rest” in Table III), right load (“R Load” in Table III), right move (“R Move” in Table III), left rest (“L Rest” in Table III), left posture (“L Posture” in Table III), left load (“L Load” in Table III), and left move (“L Move” in Table III). These additional frequency vs cycle graphs were generated using the same procedures that were used to generate FIG. 12, but were generated based on accelerometer measurement signals (of the type shown in FIG. 2) for right rest (“R Rest” in Table III), right posture (“R Posture” in Table III), right load (“R Load” in Table III), right move (“R Move” in Table III), left rest (“L Rest” in Table III), left posture (“L Posture” in Table III), left load (“L Load” in Table III), and left move (“L Move” in Table III). During left rest, an accelerometer is mounted on the patient's left hand and the patient's arm is held in a vertical position at his side. During left posture, an accelerometer is mounted on the patient's left hand and measurements are taken while the patient holds his left arm out horizontal to the ground. During left load, an accelerometer and a weight are mounted on the patient's left hand and measurements are taken while the patient's left arm is held out horizontal to the ground. During left move, an accelerometer is mounted on the patient's left hand and measurements are taken while the patient moves his left hand back and forth continuously between his nose and an extended position with his arm parallel to the ground.

In the lower part of Table III, each “Bin” number indicates a frequency from the graph of FIG. 12. The numbers to the right of each bin number indicate the number of times that particular frequency occurs in the frequency vs. cycle graph for each of right rest, right posture, right load, etc.

Table IV recites the average of right arm and left arm data in Table III. For example, the Mean “Rest” value of 7.02 in Table IV is the average of the right rest and the left rest value in Table III. The Median “Posture” value of 6.33 in Table IV is the average of the right posture and the left posture values in Table III. The value of 29.0 for Bin 5 in the “Rest” column in Table IV is the average of the right rest value of 14 in Bin 5 of Table III and the left rest value of 44 in Bin 5 of Table III.

Table V sets forth the mean, standard deviation, and median calculations for the amplitudes set forth in the amplitude vs. cycle graph of FIG. 14 (right posture, “R Posture”), as well as for additional amplitude vs. cycle graphs that were generated (but not shown here) for right rest (“R Rest” in Table V), right load (“R Load” in Table V), right move (“R Move” in Table V), left rest (“L Rest” in Table V), left posture (“L Posture” in Table V), left load (“L Load” in Table V), and left move (“L Move” in Table V). These additional frequency vs cycle graphs were generated using the same procedures that were used to generate FIG. 14, but were generated based on accelerometer measurement signals for right rest (“R Rest” in Table V), right posture (“R Posture” in Table V), right load (“R Load” in Table V), right move (“R Move” in Table V), left rest (“L Rest” in Table V), left posture (“L Posture” in Table V), left load (“L Load” in Table V), and left move (“L Move” in Table V).

In the lower part of Table V, each “Bin” number indicates one of twenty equal increments extending from the lowest to the greatest amplitude in FIG. 14. The numbers to the right of each bin number indicate the number of times that an amplitude falls within that particular increment in the amplitude vs. cycle graph for each of right rest, right posture, right load, etc.

Table VI recites the average of right arm and left arm data in Table V. For example, the Mean “Rest” value of 124.62 in Table VI is the average of the right rest and the left rest value in Table V. The Median “Posture” value of 637.15 in Table VI is the average of the right posture and the left posture values in Table V. The value of 10.0 for Bin 5 in the “Rest” column in Table VI is the average of the right rest value of 10 in Bin 5 of Table V and the left rest value of 10 in Bin 5 of Table V.

Table VII sets forth the mean, standard deviation, and median calculations for the amplitudes set forth in the area vs. cycle graph of FIG. 13 (right posture, “R Posture”), as well as for additional area vs. cycle graphs that were generated (but not shown here) for right rest (“R Rest” in Table VII), right load (“R Load” in Table VII), right move (“R Move” in Table VII), left rest (“L Rest” in Table VII), left posture (“L Posture” in Table VII), left load (“L Load” in Table VII), and left move (“L Move” in Table VII). These additional frequency vs cycle graphs were generated using the same procedures that were used to generate FIG. 13, but were generated from accelerometer measurement signals for right rest (“R Rest” in Table VII), right posture (“R Posture” in Table VII), right load (“R Load” in Table VII), right move (“R Move” in Table VII), left rest (“L Rest” in Table VII), left posture (“L Posture” in Table VII), left load (“L Load” in Table VII), and left move (“L Move” in Table VII).

In the lower part of Table VII, each “Bin” number indicates one of twenty equal increments extending from the lowest to the greatest area in FIG. 13. The numbers to the right of each bin number indicate the number of times that an area of a cycle falls within that particular increment in the area vs. cycle graph for each of right rest, right posture, right load, etc.

Table VIII recites the average of right arm and left arm data in Table VII. For example, the Mean “Rest” value of 7.07 in Table VIII is the average of the mean right rest value 1.37 and the mean left rest value 12.76 in Table VII. The Median “Posture” value of 36.90 in Table VIII is the average of the right posture and the left posture values in Table VIII. The value of 7.5 for Bin 5 in the “Rest” column in Table VIII is the average of the right rest value of 7 in Bin 5 of Table VII and the left rest value of 8 in Bin 5 of Table VII.

Tables I to VIII are generated from accelerometer measurements made for the left and/or right arm of a patient with known tremor problems, say patient “A”.

Tables IX to XIV are generated from accelerometer measurements made for the left and/or right arm of a normal control subject with no known tremor problems, say subject “B”.

Table IX below sets forth the mean, standard deviation, and median calculations for the frequencies set forth in frequency vs. cycle graphs that were generated (but not shown here) for right rest (“R Rest” in Table IX), right posture (“R Posture in Table IX), right load (“R Load” in Table IX), right move (“R Move” in Table IX), left rest (“L Rest” in Table IX), left posture (“L Posture” in Table IX), left load (“L Load” in Table IX), and left move (“L Move” in Table IX). The frequency vs cycle graphs relied on to prepare Table IX were generated using the same procedures that were used to generate FIG. 12, but were generated based on data produced from accelerometer measurement signals for right posture (“R Posture in Table IX), right rest (“R Rest” in Table IX), right load (“R Load” in Table IX), right move (“R Move” in Table IX), left rest (“L Rest” in Table IX), left posture (“L Posture” in Table IX), left load (“L Load” in Table IX), and left move (“L Move” in Table IX).

In the lower part of Table IX, each “Bin” number indicates a frequency in the range of one to 20 Hz. The numbers to the right of each bin number indicate the number of times that particular frequency occurs in the frequency vs. cycle graph for each of right rest, right posture, right load, etc.

Table X recites the average of right arm and left arm data in Table IX. For example, the Mean “Rest” value of 8.68 in Table X is the average of the right rest value of 8.30 and the left rest value of 9.05 in Table IX. The Median “Posture” value of 7.53 in Table X is the average of the right posture value of 7.75 and the left posture value of 7.30 in Table IX. The value of 3 for Bin 5 in the “Rest” column in Table X is the average of the right rest value of 3 in Bin 5 of Table IX and the left rest value of 3 in Bin 5 of Table IX.

Table XI sets forth the mean, standard deviation, and median calculations for the amplitudes set forth in additional amplitude vs. cycle graphs which are of the type shown in FIG. 14 and are generated (but not shown here) for right rest (“R Rest” in Table XI), right posture (“R posture in Table XI), right load (“R Load” in Table XI), right move (“R Move” in Table XI), left rest (“L Rest” in Table XI), left posture (“L Posture” in Table XI), left load (“L Load” in Table XI), and for left load (“L Load” in Table XI). These additional amplitude vs cycle graphs were generated using the same procedures that were used to generate FIG. 14, and were based on data produced from accelerometer measurement signals for right rest (“R Rest” in Table XI), right posture (“R Posture” in Table XI), right load (“R Load” in Table XI), right move (“R Move” in Table XI), left rest (“L Rest” in Table XI), left posture (“L Posture” in Table XI), left load (“L Load” in Table XI), and for left move (“L Move” in Table XI).

In the lower part of Table XI, each “Bin” number indicates one of twenty equal increments extending from the lowest to the greatest amplitude in the amplitude vs. cycle graphs used to generate the data. The numbers to the right of each bin number indicate the number of times that an amplitude falls within that particular increment in the amplitude vs. cycle graph for each of right rest, right posture, right load, etc.

Table XII recites the average of right arm and left arm data in Table XI. For example, the Mean “Rest” value of 27.18 in Table XII is the average of the right rest value of 29.03 and the left rest value of 25.33 in Table XI. The Median “Posture” value of 140.50 in Table XII is the average of the median right posture value of 185.00 in Table XI and the median left posture value of 96.00 in Table XI. The value of 5.50 for Bin 5 in the “Rest” column in Table XII is the average of the right rest value of 10 in Bin 5 of Table XI and the left rest value of 1 in Bin 5 of Table XI.

Table XIII sets forth the mean, standard deviation, and median calculations for the areas set forth in additional area vs. cycle graphs that were generated (but not shown here) for right posture (“R Posture” in Table XIII), right rest (“R Rest” in Table VII), right load (“R Load” in Table VII), right move (“R Move” in Table VII), left rest (“L Rest” in Table VII), left posture (“L Posture” in Table VIII), left load (“L Load” in Table VII), and for left move (“L Move” in Table VII). The additional area vs. cycle graphs were generated using the same procedures that were used to generate FIG. 13, and were generated based on data produced from accelerometer measurement signals for right posture (“R Posture” in Table XIII), right rest (“R Rest” in Table XIII), right load (“R Load” in Table XIII), right move (“R Move” in Table XIII), left rest (“L Rest” in Table XIII), left posture (“L Posture” in Table XIII), left load (“L Load” in Table XIII), and for left move (“L Move” in Table XIII).

In the lower part of Table XIII, each “Bin” number indicates one of twenty equal increments extending from the lowest to the greatest area in the area vs. cycle graph from which the data is taken. The numbers to the right of each bin number indicate the number of times that an area of a cycle falls within that particular increment in the area vs. cycle graph for each of right rest, right posture, right load, etc.

Table XIV recites the average of right arm and left arm data in Table XIII in the same manner as Table XII, Table X, etc.

FIG. 19 is a graphical depiction of mean and standard deviation data from Table IV.

FIG. 20 is a graphical depiction of the “Bin” data from Table IV.

FIG. 21 is a graphical depiction of mean and standard deviation data from Table VI.

FIG. 22 is a graphical depiction of “Bin” data from Table VI.

FIG. 23 is a graphical depiction of mean and standard deviation data from Table VIII.

FIG. 24 is a graphical depiction of “Bin” data from Table VIII.

FIG. 25 is a graphical depiction of mean and standard deviation data from Table X.

FIG. 26 is a graphical depiction of “Bin” data from Table X.

FIG. 27 is a graphical depiction of mean and standard deviation data data from Table XII.

FIG. 28 is a graphical depiction of “Bin” data from Table XII.

FIG. 29 is a graphical depiction of mean and standard deviation data from Table XIV.

FIG. 30 is a graphical depiction of “Bin” data from Table XIV.

The following examples are presented by way of illustration, and not limitation, of the invention.

### EXAMPLE I

An accelerometer is mounted on the right hand of a first patient. In step 20 of FIG. 31, accelerometer readings of the type illustrated in FIG. 2 are produced and used during various tremor tests to generate an oscillating signal of the type shown in FIG. 3A. An oscillating signal of the type shown in FIG. 3A is first produced for each axis 11 to 13 during testing when the right hand of the patient is at rest, i.e., for “Right Rest”. The procedure is repeated for “Right Posture” test (right arm held horizontal to the ground with accelerometer mounted on right hand) and an oscillating signal of the type shown in FIG. 3A is produced for each axis 11 to 13. The procedure is repeated for “Right Load” test (right arm held horizontal to the ground with accelerometer and a load mounted on the right hand). The procedure is repeated for “Right Move” test (right hand moving between nose and extended with arm horizontal to ground with accelerometer mounted on right hand) and an oscillating signal of the type shown in FIG. 3A is produced for each axis 11 to 13. The accelerometer is then removed and mounted on the left hand and oscillating signals are obtained for “Left Rest” test, “Left Posture” test, “Left Load” test, and “Left Move” test. In step 21 of FIG. 31, each oscillating signal produced is subjected to the min/max procedure illustrated in FIGS. 3A and 3B to remove or minimize the effect of rotation and orientation. As a result, three signals (one signal for U/D, one for R/L, and one for F/B) of the type shown in FIGS. 4C, 5C, 6C are produced for the “Right Rest” test, three signals are produced for “Right Posture” test, three for “Right Load” test, etc. The three signals for each test are combined (step 22) using the sum of the squares—square root procedure earlier described in connection with FIGS. 4D, 5D, 6D, 7A, 7B to produce a composite signal of the type shown in FIGS. 7C and 8. Consequently, each test—whether it be the right rest test, left rest test, etc.—produces a composite signal of the type shown in FIGS. 7C and 8.

The composite signal for each test is analyzed (step 23). This analysis is carried out by examining, in the manner discussed with respect to FIG. 11, each cycle comprising a composite signal. After the cycle analysis of a composite signal is complete, data of the type set forth in Tables I and II is available. When this data is available, graphical representations of the type shown in FIGS. 12 to 30 are prepared (step 24).

### EXAMPLE II

The tremor classification graphical representation illustrated in FIG. 18 includes a section from about 1 cycle to 31 cycles which indicates no tremor; includes a section from about 31 cycles to 48 cycles which indicates a “burst” type of tremor (step 25) with a magnitude over 3.5 on the vertical “combined freq-area” axis; includes a section from about 48 cycles to 68 cycles which indicates a “steady” type of tremor with a magnitude of 2.0 to 2.5 on the vertical axis after a burst; includes a section from about 68 to 91 cycles which indicates no tremor; includes a section from about 91 to 98 cycles which indicates a “steady” type of tremor with a magnitude of 2.0 to 2.5 on the vertical axis; and, includes a section from about 98 to 116 cycles which indicates a “burst” type of tremor with a magnitude over 3.5 on the vertical “combined freq-area” axis.

Five hundred patients with Parkinson's disease are tested for tremor. Three hundred of the patients are male with an age in the range of 20 to 50. Two hundred of the patients are female with an age in the range of 20 to 50. Each patient is tested for right rest, right posture, right move, right load, left rest, left posture, left load, and left move. For each test the accelerometer measurements were processed in the manner described for FIGS. 1 to 18, and a graph comparable to FIG. 18 is produced. For right posture, 88% of the patients tested have a FIG. 18 graph which has a “burst” greater than 3.5 on the “Combined Freq. And Area” vertical scale, followed by a “steady” in the range of 2.0 to 2.5 on the vertical scale. Consequently, this combination of a “burst” and a “steady” suggests with a high probability that a patient is suffering from Parkinson's disease. 84% of the patients tested for right posture also had a frequency vs. cycle graph of the type shown in FIG. 17 in which there was a group of at least fifteen consecutive cycles comparable to the group of 31 to 48 cycles in FIG. 17 in which the area for each consecutive cycle was greater than the area of 6000 on the vertical axis. Consequently, an area vs. cycle graph of the type shown in FIG. 17 in which there are at least fifteen consecutive cycles with an area over 6000 indicates with a high probability that patient has Parkinson's disease.

### EXAMPLE III

In FIG. 19 (frequency), a low standard deviation indicates that there is tremor.

### EXAMPLE IV

In FIG. 20 (frequency), the frequencies are concentrated between 4 and 9 Hz, which indicates tremor.

### EXAMPLE V

In FIG. 21 (amplitude), a high standard deviation indicates tremor.

### EXAMPLE VI

In FIG. 22 (amplitude), the wide range of amplitudes indicates tremor.

TABLE I

| | Record r4p3 | | | | |
|---|---|---|---|---|---|
| Cycle Values | Test | | Right Posture | | |
| Cycle | Start | Period | Freq. | Amplitude | Area |
| 0 | 62 | 108 | 9.26 | 262 | 86 |
| 1 | 170 | 123 | 8.13 | 249 | 100 |
| 2 | 293 | 107 | 9.35 | 207 | 60 |
| 3 | 400 | 117 | 8.55 | 109 | 40 |
| 4 | 517 | 113 | 8.85 | 133 | 42 |
| 5 | 630 | 118 | 8.47 | 113 | 36 |
| 6 | 748 | 122 | 8.20 | 226 | 89 |
| 7 | 870 | 145 | 6.90 | 371 | 168 |
| 8 | 1015 | 143 | 6.99 | 318 | 150 |
| 9 | 1158 | 117 | 8.55 | 231 | 84 |
| 10 | 1275 | 233 | 4.29 | 188 | 113 |
| 11 | 1508 | 142 | 7.04 | 199 | 83 |
| 12 | 1650 | 93 | 10.75 | 216 | 54 |
| 13 | 1743 | 130 | 7.69 | 170 | 57 |
| 14 | 1873 | 125 | 8.00 | 326 | 99 |
| 15 | 1998 | 157 | 6.37 | 226 | 100 |
| 16 | 2155 | 120 | 8.33 | 286 | 103 |
| 17 | 2275 | 109 | 9.17 | 253 | 73 |
| 18 | 2384 | 136 | 7.35 | 229 | 93 |
| 19 | 2520 | 122 | 8.20 | 453 | 157 |
| 20 | 2642 | 146 | 6.85 | 449 | 158 |
| 21 | 2788 | 212 | 4.72 | 481 | 260 |
| 22 | 3000 | 145 | 6.90 | 402 | 143 |
| 23 | 3145 | 195 | 5.13 | 228 | 119 |
| 24 | 3340 | 115 | 8.70 | 192 | 67 |
| 25 | 3455 | 102 | 9.80 | 249 | 85 |
| 26 | 3557 | 125 | 8.00 | 404 | 98 |
| 27 | 3682 | 112 | 8.93 | 461 | 134 |
| 28 | 3794 | 73 | 13.70 | 182 | 44 |
| 29 | 3867 | 100 | 10.00 | 390 | 124 |
| 30 | 3967 | 72 | 13.89 | 251 | 51 |
| 31 | 4039 | 112 | 8.93 | 463 | 156 |
| 32 | 4151 | 88 | 11.36 | 209 | 64 |
| 33 | 4239 | 131 | 7.63 | 348 | 97 |
| 34 | 4370 | 176 | 5.68 | 463 | 153 |
| 35 | 4546 | 180 | 5.56 | 521 | 221 |
| 36 | 4726 | 96 | 10.42 | 503 | 141 |
| 37 | 4822 | 95 | 10.53 | 442 | 105 |
| 38 | 4917 | 138 | 7.25 | 685 | 287 |
| 39 | 5055 | 167 | 5.99 | 720 | 372 |
| 40 | 5222 | 158 | 6.33 | 800 | 321 |

TABLE II

| | Record: r4p3 | | | | | |
|---|---|---|---|---|---|---|
| Cycle Details | Test | | Right Posture | | | |
| Cycle | Pos | Neg | Maximum | Minimum | Above | Below |
| 0 | 51 | 57 | 132 | −130 | 39 | 47 |
| 1 | 49 | 74 | 126 | −123 | 37 | 63 |
| 2 | 58 | 49 | 114 | −93 | 35 | 25 |
| 3 | 65 | 52 | 61 | −48 | 23 | 16 |
| 4 | 49 | 64 | 66 | −67 | 20 | 22 |
| 5 | 71 | 47 | 55 | −58 | 21 | 14 |
| 6 | 42 | 80 | 84 | −142 | 21 | 68 |
| 7 | 52 | 93 | 175 | −196 | 53 | 115 |
| 8 | 89 | 54 | 185 | −133 | 106 | 44 |
| 9 | 61 | 56 | 117 | −114 | 43 | 41 |
| 10 | 41 | 192 | 114 | −74 | 26 | 87 |
| 11 | 83 | 59 | 97 | −102 | 42 | 41 |
| 12 | 41 | 52 | 104 | −112 | 25 | 28 |
| 13 | 89 | 41 | 113 | −57 | 43 | 14 |
| 14 | 47 | 78 | 156 | −170 | 40 | 59 |
| 15 | 95 | 62 | 136 | −90 | 66 | 34 |
| 16 | 70 | 50 | 133 | −153 | 54 | 50 |
| 17 | 49 | 60 | 131 | −122 | 36 | 37 |
| 18 | 67 | 69 | 134 | −95 | 54 | 39 |
| 19 | 70 | 52 | 210 | −243 | 74 | 83 |
| 20 | 56 | 90 | 233 | −216 | 76 | 82 |
| 21 | 142 | 70 | 225 | −256 | 154 | 106 |
| 22 | 52 | 93 | 232 | −170 | 73 | 71 |
| 23 | 125 | 70 | 133 | −95 | 71 | 48 |
| 24 | 41 | 74 | 96 | −96 | 23 | 43 |
| 25 | 39 | 63 | 80 | −169 | 17 | 67 |
| 26 | 40 | 85 | 164 | −240 | 38 | 61 |
| 27 | 45 | 67 | 253 | −208 | 64 | 70 |
| 28 | 36 | 37 | 50 | −132 | 13 | 30 |
| 29 | 45 | 55 | 199 | −191 | 52 | 72 |
| 30 | 34 | 38 | 98 | −153 | 17 | 34 |
| 31 | 46 | 66 | 245 | −218 | 69 | 87 |
| 32 | 51 | 37 | 106 | −103 | 40 | 25 |
| 33 | 37 | 94 | 136 | −212 | 30 | 67 |
| 34 | 51 | 125 | 255 | −208 | 75 | 77 |
| 35 | 121 | 59 | 239 | −282 | 113 | 108 |
| 36 | 49 | 47 | 288 | −215 | 86 | 55 |
| 37 | 40 | 55 | 121 | −321 | 26 | 79 |
| 38 | 45 | 93 | 346 | −339 | 92 | 194 |
| 39 | 65 | 102 | 341 | −379 | 126 | 246 |
| 40 | 84 | 74 | 403 | −397 | 154 | 167 |

TABLE III
Frequency

| Frequency | R Rest | R Posture | R Load | R Move | L Rest | L Posture | L Load | L Move |
|---|---|---|---|---|---|---|---|---|
| Mean | 7.69 | 7.07 | 6.25 | 9.18 | 6.34 | 6.77 | 7.29 | 9.25 |
| Std Dev | 3.05 | 2.12 | 1.41 | 2.78 | 2.40 | 1.93 | 1.22 | 2.31 |
| Median | 6.71 | 6.29 | 6.17 | 8.70 | 5.08 | 6.37 | 7.14 | 9.01 |
| Frequency | | | | | | | | |
| Bin | R Rest | R Posture | R Load | R Move | L Rest | L Posture | L Load | L Move |
| 1 | 0 | 0 | 0 | 0 | 0 | 0 | 0 | 0 |
| 2 | 0 | 0 | 0 | 0 | 0 | 0 | 0 | 1 |
| 3 | 1 | 0 | 0 | 0 | 0 | 0 | 0 | 0 |
| 4 | 2 | 1 | 3 | 1 | 4 | 2 | 0 | 0 |
| 5 | 14 | 2 | 8 | 4 | 44 | 5 | 1 | 1 |
| 6 | 24 | 38 | 29 | 6 | 10 | 28 | 8 | 2 |
| 7 | 12 | 24 | 43 | 9 | 7 | 35 | 33 | 12 |
| 8 | 10 | 10 | 8 | 17 | 5 | 12 | 44 | 13 |
| 9 | 7 | 10 | 5 | 19 | 8 | 5 | 8 | 19 |
| 10 | 7 | 4 | 0 | 12 | 16 | 6 | 3 | 19 |
| 11 | 7 | 5 | 2 | 9 | 4 | 2 | 3 | 13 |
| 12 | 4 | 1 | 0 | 6 | 2 | 2 | 0 | 8 |
| 13 | 6 | 1 | 1 | 8 | 0 | 2 | 0 | 5 |
| 14 | 3 | 2 | 1 | 1 | 0 | 0 | 1 | 2 |
| 15 | 1 | 0 | 0 | 4 | 1 | 0 | 0 | 3 |
| 16 | 1 | 1 | 0 | 2 | 0 | 0 | 0 | 1 |
| 17 | 1 | 0 | 0 | 2 | 0 | 0 | 0 | 1 |
| 18 | 1 | 0 | 0 | 0 | 0 | 0 | 0 | 0 |
| 19 | 0 | 0 | 0 | 0 | 0 | 1 | 0 | 0 |
| 20 | 0 | 0 | 0 | 0 | 0 | 0 | 0 | 0 |

TABLE IV
Frequency

Test Average

| | Rest | Posture | Load | Move |
|---|---|---|---|---|
| Mean | 7.02 | 6.92 | 6.77 | 9.22 |
| Std Dev | 2.73 | 2.03 | 1.32 | 2.55 |
| Median | 5.90 | 6.33 | 6.66 | 8.86 |

Test Average

| Bin | Rest | Posture | Load | Move |
|---|---|---|---|---|
| 1 | 0.0 | 0.0 | 0.0 | 0.0 |
| 2 | 0.0 | 0.0 | 0.0 | 0.5 |
| 3 | 0.5 | 0.0 | 0.0 | 0.0 |
| 4 | 3.0 | 1.5 | 1.5 | 0.5 |
| 5 | 29.0 | 3.5 | 4.5 | 2.5 |
| 6 | 17.0 | 33.0 | 18.5 | 4.0 |
| 7 | 9.5 | 29.5 | 38.0 | 10.5 |
| 8 | 7.5 | 11.0 | 26.0 | 15.0 |
| 9 | 7.5 | 7.5 | 6.5 | 19.0 |
| 10 | 11.5 | 5.0 | 1.5 | 15.5 |
| 11 | 5.5 | 3.5 | 2.5 | 11.0 |
| 12 | 3.0 | 1.5 | 0.0 | 7.0 |
| 13 | 3.0 | 1.5 | 0.5 | 6.5 |
| 14 | 1.5 | 1.0 | 1.0 | 1.5 |
| 15 | 1.0 | 0.0 | 0.0 | 3.5 |
| 16 | 0.5 | 0.5 | 0.0 | 1.5 |
| 17 | 0.5 | 0.0 | 0.0 | 1.5 |
| 18 | 0.5 | 0.0 | 0.0 | 0.0 |
| 19 | 0.0 | 0.5 | 0.0 | 0.0 |
| 20 | 0.0 | 0.0 | 0.0 | 0.0 |

TABLE V
Amplitude

| Amplitude | R Rest | R Posture | R Load | R Move | L Rest | L Posture | L Load | L Move |
|---|---|---|---|---|---|---|---|---|
| Mean | 30.39 | 885.85 | 377.56 | 337.83 | 218.85 | 586.07 | 279.54 | 533.70 |
| Std Dev | 23.75 | 257.60 | 185.45 | 297.25 | 195.50 | 522.00 | 206.00 | 166.70 |
| Median | 12.61 | 777.38 | 170.67 | 187.99 | 106.10 | 496.92 | 135.70 | 342.88 |
| Amplitude | | | | | | | | |
| Bin | R Rest | R Posture | R Load | R Move | L Rest | L Posture | L Load | L Move |
| 1 | 0 | 2 | 0 | 2 | 0 | 10 | 0 | 2 |
| 2 | 0 | 18 | 1 | 9 | 1 | 10 | 1 | 13 |
| 3 | 0 | 20 | 1 | 8 | 5 | 12 | 1 | 14 |
| 4 | 6 | 14 | 11 | 8 | 8 | 10 | 8 | 12 |
| 5 | 10 | 10 | 8 | 16 | 10 | 20 | 13 | 9 |
| 6 | 5 | 8 | 8 | 14 | 16 | 7 | 13 | 15 |
| 7 | 9 | 1 | 7 | 11 | 16 | 7 | 15 | 9 |
| 8 | 9 | 4 | 4 | 6 | 10 | 2 | 7 | 8 |
| 9 | 10 | 1 | 9 | 8 | 13 | 2 | 11 | 6 |
| 10 | 11 | 1 | 9 | 4 | 4 | 2 | 8 | 3 |
| 11 | 10 | 2 | 3 | 5 | 6 | 0 | 1 | 2 |
| 12 | 6 | 2 | 9 | 4 | 1 | 2 | 8 | 1 |
| 13 | 7 | 2 | 3 | 2 | 0 | 2 | 5 | 3 |
| 14 | 4 | 0 | 5 | 1 | 2 | 2 | 2 | 1 |
| 15 | 4 | 6 | 9 | 0 | 2 | 2 | 0 | 0 |
| 16 | 2 | 2 | 6 | 1 | 3 | 3 | 1 | 0 |
| 17 | 1 | 1 | 5 | 0 | 2 | 2 | 1 | 1 |
| 18 | 2 | 1 | 1 | 0 | 1 | 2 | 3 | 1 |
| 19 | 1 | 1 | 0 | 0 | 0 | 2 | 1 | 0 |
| 20 | 1 | 1 | 1 | 1 | 1 | 1 | 3 | 1 |

TABLE VI
Amplitude

Test Average

| | Rest | Posture | Load | Move |
|---|---|---|---|---|
| Mean | 124.62 | 735.96 | 328.55 | 435.77 |
| Std Dev | 109.63 | 389.80 | 195.73 | 231.98 |
| Median | 59.36 | 637.15 | 153.19 | 265.44 |

Test Average

| Bin | Rest | Posture | Load | Move |
|---|---|---|---|---|
| 1 | 0.0 | 6.0 | 0.0 | 2.0 |
| 2 | 0.5 | 14.0 | 1.0 | 11.0 |
| 3 | 2.5 | 16.0 | 1.0 | 11.0 |
| 4 | 7.0 | 12.0 | 9.5 | 10.0 |
| 5 | 10.0 | 15.0 | 10.5 | 12.5 |
| 6 | 10.5 | 7.5 | 10.5 | 14.5 |
| 7 | 12.5 | 4.0 | 11.0 | 10.0 |
| 8 | 9.5 | 3.0 | 5.5 | 7.0 |
| 9 | 11.5 | 1.5 | 10.0 | 7.0 |
| 10 | 7.5 | 1.5 | 8.5 | 3.5 |
| 11 | 8.0 | 1.0 | 2.0 | 3.5 |
| 12 | 3.5 | 2.0 | 8.5 | 2.5 |
| 13 | 3.5 | 2.0 | 4.0 | 2.5 |
| 14 | 3.0 | 1.0 | 3.5 | 1.0 |
| 15 | 3.0 | 4.0 | 4.5 | 0.0 |
| 16 | 2.5 | 2.5 | 3.5 | 0.5 |
| 17 | 1.5 | 1.5 | 3.0 | 0.5 |
| 18 | 1.5 | 1.5 | 2.0 | 0.5 |
| 19 | 0.5 | 1.5 | 0.5 | 0.0 |
| 20 | 1.0 | 1.0 | 2.0 | 1.0 |

TABLE VII
Area

| Area | R Rest | R Posture | R Load | R Move | L Rest | L Posture | L Load | L Move |
|---|---|---|---|---|---|---|---|---|
| Mean | 1.37 | 44.79 | 20.17 | 11.46 | 12.76 | 30.03 | 11.89 | 19.57 |
| Std Dev | 0.24 | 15.18 | 27.13 | 6.72 | 6.35 | 9.20 | 6.09 | 6.33 |
| Median | 0.90 | 45.91 | 11.45 | 8.49 | 8.40 | 27.88 | 6.14 | 27.84 |
| Area | | | | | | | | |
| Bin | R Rest | R Posture | R Load | R Move | L Rest | L Posture | L Load | L Move |
| 1 | 2 | 16 | 1 | 14 | 3 | 12 | 1 | 57 |
| 2 | 13 | 24 | 2 | 20 | 7 | 13 | 0 | 34 |
| 3 | 14 | 13 | 7 | 23 | 13 | 12 | 5 | 6 |
| 4 | 10 | 9 | 10 | 15 | 16 | 11 | 10 | 2 |
| 5 | 7 | 9 | 9 | 11 | 8 | 12 | 12 | 1 |
| 6 | 11 | 4 | 8 | 12 | 8 | 8 | 12 | 0 |
| 7 | 8 | 4 | 6 | 2 | 11 | 5 | 10 | 0 |
| 8 | 7 | 1 | 7 | 2 | 9 | 1 | 14 | 0 |
| 9 | 7 | 1 | 7 | 0 | 13 | 3 | 8 | 0 |
| 10 | 8 | 2 | 7 | 0 | 2 | 4 | 5 | 0 |
| 11 | 4 | 1 | 2 | 0 | 2 | 0 | 3 | 0 |
| 12 | 1 | 3 | 4 | 0 | 3 | 1 | 5 | 0 |
| 13 | 1 | 3 | 4 | 1 | 1 | 2 | 4 | 0 |
| 14 | 4 | 1 | 3 | 0 | 0 | 2 | 1 | 0 |
| 15 | 0 | 2 | 3 | 0 | 0 | 2 | 2 | 0 |
| 16 | 1 | 2 | 6 | 0 | 2 | 1 | 1 | 0 |
| 17 | 0 | 3 | 6 | 0 | 0 | 3 | 2 | 0 |
| 18 | 0 | 0 | 3 | 0 | 3 | 0 | 1 | 0 |
| 19 | 1 | 2 | 4 | 0 | 1 | 5 | 1 | 0 |
| 20 | 1 | 1 | 2 | 1 | 0 | 3 | 2 | 1 |

TABLE VIII
Area

Test Average

| | Rest | Posture | Load | Move |
|---|---|---|---|---|
| Mean | 7.07 | 37.41 | 16.03 | 15.52 |
| Std Dev | 3.30 | 12.19 | 16.61 | 6.53 |
| Median | 4.65 | 36.90 | 8.80 | 18.17 |

Test Average

| Bin | Rest | Posture | Load | Move |
|---|---|---|---|---|
| 1 | 2.5 | 14.0 | 1.0 | 35.5 |
| 2 | 10.0 | 18.5 | 1.0 | 27.0 |
| 3 | 13.5 | 12.5 | 6.0 | 14.5 |
| 4 | 13.0 | 10.0 | 10.0 | 8.5 |
| 5 | 7.5 | 10.5 | 10.5 | 6.0 |
| 6 | 9.5 | 6.0 | 10.0 | 6.0 |
| 7 | 9.5 | 4.5 | 8.0 | 1.0 |
| 8 | 8.0 | 1.0 | 10.5 | 1.0 |
| 9 | 10.0 | 2.0 | 7.5 | 0.0 |
| 10 | 5.0 | 3.0 | 6.0 | 0.0 |
| 11 | 3.0 | 0.5 | 2.5 | 0.0 |
| 12 | 2.0 | 2.0 | 4.5 | 0.0 |
| 13 | 1.0 | 2.5 | 4.0 | 0.5 |
| 14 | 2.0 | 1.5 | 2.0 | 0.0 |
| 15 | 0.0 | 2.0 | 2.5 | 0.0 |
| 16 | 1.5 | 1.5 | 3.5 | 0.0 |
| 17 | 0.0 | 3.0 | 4.0 | 0.0 |
| 18 | 1.5 | 0.0 | 2.0 | 0.0 |
| 19 | 1.0 | 3.5 | 2.5 | 0.0 |
| 20 | 0.5 | 2.0 | 2.0 | 1.0 |

TABLE IX
Frequency

| Frequency | R Rest | R Posture | R Load | R Move | L Rest | L Posture | L Load | L Move |
|---|---|---|---|---|---|---|---|---|
| Mean | 8.30 | 8.10 | 8.87 | 8.98 | 9.05 | 8.68 | 9.65 | 8.76 |
| Std Dev | 2.64 | 2.03 | 2.65 | 2.98 | 2.79 | 3.06 | 3.04 | 2.90 |
| Median | 7.58 | 7.75 | 8.00 | 8.73 | 8.73 | 7.30 | 8.85 | 8.13 |
| Frequency | | | | | | | | |
| 0 | R Rest | R Posture | R Load | R Move | L Rest | L Posture | L Load | L Move |
| 1 | 0 | 0 | 0 | 0 | 0 | 0 | 0 | 0 |
| 2 | 0 | 0 | 0 | 0 | 0 | 0 | 0 | 0 |
| 3 | 0 | 0 | 0 | 0 | 1 | 0 | 0 | 0 |
| 4 | 1 | 0 | 0 | 2 | 2 | 0 | 1 | 0 |
| 5 | 3 | 1 | 1 | 6 | 3 | 0 | 2 | 4 |
| 6 | 11 | 6 | 4 | 10 | 8 | 5 | 4 | 11 |
| 7 | 19 | 21 | 15 | 13 | 10 | 35 | 9 | 15 |
| 8 | 24 | 28 | 29 | 11 | 15 | 27 | 19 | 18 |
| 9 | 14 | 21 | 18 | 11 | 16 | 6 | 17 | 13 |
| 10 | 7 | 8 | 8 | 14 | 11 | 2 | 11 | 13 |
| 11 | 5 | 7 | 7 | 6 | 13 | 3 | 9 | 9 |
| 12 | 3 | 3 | 4 | 7 | 8 | 2 | 6 | 4 |
| 13 | 5 | 1 | 4 | 9 | 5 | 5 | 7 | 3 |
| 14 | 3 | 1 | 1 | 4 | 4 | 4 | 3 | 3 |
| 15 | 3 | 1 | 4 | 6 | 2 | 6 | 5 | 1 |
| 16 | 1 | 1 | 3 | 1 | 1 | 2 | 3 | 2 |
| 17 | 1 | 0 | 1 | 0 | 1 | 2 | 3 | 3 |
| 18 | 0 | 1 | 1 | 0 | 1 | 1 | 1 | 1 |
| 19 | 0 | 0 | 0 | 0 | 0 | 0 | 1 | 0 |
| 20 | 0 | 0 | 0 | 0 | 0 | 0 | 0 | 0 |

TABLE X
Frequency

Test Average

| | Rest | Posture | Load | Move |
|---|---|---|---|---|
| Mean | 8.68 | 8.39 | 9.26 | 8.87 |
| Std Dev | 2.72 | 2.55 | 2.85 | 2.94 |
| Median | 8.16 | 7.53 | 8.43 | 8.43 |

Test Average

| Bin | Rest | Posture | Load | Move |
|---|---|---|---|---|
| 1 | 0.0 | 0.0 | 0.0 | 0.0 |
| 2 | 0.0 | 0.0 | 0.0 | 0.0 |
| 3 | 0.5 | 0.0 | 0.0 | 0.0 |
| 4 | 1.5 | 0.0 | 0.5 | 1.0 |
| 5 | 3.0 | 0.5 | 1.5 | 5.0 |
| 6 | 9.5 | 5.5 | 4.0 | 10.5 |
| 7 | 14.5 | 28.0 | 12.0 | 14.0 |
| 8 | 19.5 | 27.5 | 24.0 | 14.5 |
| 9 | 15.0 | 13.5 | 17.5 | 12.0 |
| 10 | 9.0 | 5.0 | 9.5 | 13.5 |
| 11 | 9.0 | 5.0 | 8.0 | 7.5 |
| 12 | 5.5 | 2.5 | 5.0 | 5.5 |
| 13 | 5.0 | 3.0 | 5.5 | 6.0 |
| 14 | 3.5 | 2.5 | 2.0 | 3.5 |
| 15 | 2.5 | 3.5 | 4.5 | 3.5 |
| 16 | 1.0 | 1.5 | 3.0 | 1.5 |
| 17 | 1.0 | 1.0 | 2.0 | 1.5 |
| 18 | 0.5 | 1.0 | 1.0 | 0.5 |
| 19 | 0.0 | 0.0 | 0.5 | 0.0 |
| 20 | 0.0 | 0.0 | 0.0 | 0.0 |

TABLE XI
Amplitude

| Amplitude | R Rest | R Posture | R Load | R Move | L Rest | L Posture | L Load | L Move |
|---|---|---|---|---|---|---|---|---|
| Mean | 29.03 | 212.78 | 94.50 | 882.27 | 25.33 | 95.94 | 68.36 | 994.98 |
| Std Dev | 12.95 | 118.49 | 40.25 | 399.55 | 34.39 | 32.18 | 24.80 | 399.64 |
| Median | 28.00 | 185.00 | 88.00 | 899.50 | 15.00 | 96.00 | 64.00 | 972.00 |
| Amplitude | R Rest | R Posture | R Load | R Move | L Rest | L Posture | L Load | L Move |
| 1 | 0 | 1 | 0 | 1 | 24 | 0 | 0 | 0 |
| 2 | 0 | 6 | 0 | 4 | 56 | 0 | 0 | 1 |
| 3 | 6 | 10 | 4 | 4 | 8 | 0 | 1 | 4 |
| 4 | 14 | 17 | 3 | 3 | 2 | 1 | 2 | 6 |
| 5 | 10 | 14 | 8 | 2 | 1 | 4 | 11 | 5 |
| 6 | 17 | 12 | 6 | 9 | 1 | 4 | 16 | 9 |
| 7 | 12 | 13 | 14 | 4 | 2 | 7 | 15 | 13 |
| 8 | 19 | 10 | 9 | 8 | 0 | 7 | 12 | 10 |
| 9 | 8 | 5 | 10 | 4 | 1 | 13 | 17 | 11 |
| 10 | 5 | 3 | 3 | 9 | 0 | 7 | 8 | 8 |
| 11 | 4 | 1 | 9 | 12 | 1 | 11 | 8 | 11 |
| 12 | 0 | 3 | 9 | 7 | 1 | 13 | 4 | 8 |
| 13 | 1 | 2 | 7 | 9 | 0 | 6 | 2 | 6 |
| 14 | 2 | 0 | 6 | 7 | 0 | 11 | 1 | 2 |
| 15 | 0 | 3 | 2 | 4 | 1 | 3 | 1 | 1 |
| 16 | 1 | 1 | 4 | 3 | 1 | 6 | 0 | 1 |
| 17 | 0 | 0 | 2 | 2 | 1 | 4 | 1 | 1 |
| 18 | 1 | 0 | 1 | 3 | 0 | 1 | 0 | 1 |
| 19 | 0 | 0 | 1 | 2 | 0 | 1 | 1 | 0 |
| 20 | 1 | 1 | 2 | 2 | 0 | 1 | 1 | 1 |

TABLE XII
Amplitude

Test Average

| | Rest | Posture | Load | Move |
|---|---|---|---|---|
| Mean | 27.18 | 154.36 | 81.43 | 938.63 |
| Std Dev | 23.67 | 75.34 | 32.53 | 399.60 |
| Median | 21.50 | 140.50 | 76.00 | 935.75 |

Test Average

| Bin | Rest | Posture | Load | Move |
|---|---|---|---|---|
| 1 | 12.0 | 0.5 | 0.0 | 0.5 |
| 2 | 28.0 | 3.0 | 0.0 | 2.5 |
| 3 | 7.0 | 5.0 | 2.5 | 4.0 |
| 4 | 8.0 | 9.0 | 2.5 | 4.5 |
| 5 | 5.5 | 9.0 | 9.5 | 3.5 |
| 6 | 9.0 | 8.0 | 11.0 | 9.0 |
| 7 | 7.0 | 10.0 | 14.5 | 8.5 |
| 8 | 9.5 | 8.5 | 10.5 | 9.0 |
| 9 | 4.5 | 9.0 | 13.5 | 7.5 |
| 10 | 2.5 | 5.0 | 5.5 | 8.5 |
| 11 | 2.5 | 6.0 | 8.5 | 11.5 |
| 12 | 0.5 | 8.0 | 6.5 | 7.5 |
| 13 | 0.5 | 4.0 | 4.5 | 7.5 |
| 14 | 1.0 | 5.5 | 3.5 | 4.5 |
| 15 | 0.5 | 3.0 | 1.5 | 2.5 |
| 16 | 1.0 | 3.5 | 2.0 | 2.0 |
| 17 | 0.5 | 2.0 | 1.5 | 1.5 |
| 18 | 0.5 | 0.5 | 0.5 | 2.0 |
| 19 | 0.0 | 0.5 | 1.0 | 1.0 |
| 20 | 0.5 | 1.0 | 1.5 | 1.5 |

TABLE XIII
Area

| Area | R Rest | R Posture | R Load | R Move | L Rest | L Posture | L Load | L Move |
|---|---|---|---|---|---|---|---|---|
| Mean | 1.11 | 8.61 | 3.42 | 31.84 | 0.98 | 3.50 | 2.16 | 35.96 |
| Std Dev | 0.64 | 5.87 | 1.89 | 21.07 | 1.67 | 1.80 | 1.15 | 19.26 |
| Median | 1.04 | 7.33 | 3.09 | 27.59 | 0.49 | 3.37 | 1.89 | 32.74 |
| Area | R Rest | R Posture | R Load | R Move | L Rest | L Posture | L Load | L Move |
| 1 | 0 | 3 | 0 | 6 | 44 | 0 | 0 | 1 |
| 2 | 10 | 18 | 5 | 9 | 41 | 3 | 1 | 6 |
| 3 | 12 | 15 | 7 | 12 | 5 | 7 | 7 | 8 |
| 4 | 10 | 15 | 11 | 11 | 1 | 10 | 13 | 9 |
| 5 | 12 | 14 | 7 | 14 | 1 | 9 | 11 | 10 |
| 6 | 12 | 9 | 7 | 12 | 0 | 9 | 13 | 10 |
| 7 | 15 | 11 | 11 | 10 | 1 | 5 | 11 | 15 |
| 8 | 12 | 3 | 8 | 6 | 1 | 9 | 7 | 6 |
| 9 | 5 | 2 | 7 | 5 | 0 | 9 | 10 | 4 |
| 10 | 3 | 3 | 8 | 4 | 0 | 6 | 6 | 11 |
| 11 | 5 | 1 | 5 | 6 | 1 | 10 | 4 | 5 |
| 12 | 1 | 2 | 5 | 2 | 2 | 8 | 4 | 3 |
| 13 | 1 | 1 | 5 | 0 | 0 | 2 | 4 | 6 |
| 14 | 1 | 1 | 4 | 1 | 0 | 5 | 2 | 1 |
| 15 | 0 | 0 | 3 | 1 | 1 | 2 | 1 | 2 |
| 16 | 1 | 1 | 4 | 1 | 0 | 1 | 1 | 0 |
| 17 | 0 | 0 | 2 | 1 | 1 | 2 | 1 | 2 |
| 18 | 1 | 0 | 0 | 1 | 1 | 0 | 0 | 0 |
| 19 | 1 | 0 | 1 | 0 | 0 | 1 | 1 | 0 |
| 20 | 1 | 1 | 2 | 0 | 1 | 2 | 2 | 1 |

TABLE XIV
Area

Test Average

| | Rest | Posture | Load | Move |
|---|---|---|---|---|
| Mean | 1.05 | 6.06 | 2.79 | 33.90 |
| Std Dev | 1.16 | 3.84 | 1.52 | 20.17 |
| Median | 0.77 | 5.35 | 2.49 | 30.17 |

Test Average

| Bin | Rest | Posture | Load | Move |
|---|---|---|---|---|
| 1 | 22.0 | 1.5 | 0.0 | 3.5 |
| 2 | 25.5 | 10.5 | 3.0 | 7.5 |
| 3 | 8.5 | 11.0 | 7.0 | 10.0 |
| 4 | 5.5 | 12.5 | 12.0 | 10.0 |
| 5 | 6.5 | 11.5 | 9.0 | 12.0 |
| 6 | 6.0 | 9.0 | 10.0 | 11.0 |
| 7 | 8.0 | 8.0 | 11.0 | 12.5 |
| 8 | 6.5 | 6.0 | 7.5 | 6.0 |
| 9 | 2.5 | 5.5 | 8.5 | 4.5 |
| 10 | 1.5 | 4.5 | 7.0 | 7.5 |
| 11 | 3.0 | 5.5 | 4.5 | 5.5 |
| 12 | 1.5 | 5.0 | 4.5 | 2.5 |
| 13 | 0.5 | 1.5 | 4.5 | 3.0 |
| 14 | 0.5 | 3.0 | 3.0 | 1.0 |
| 15 | 0.5 | 1.0 | 2.0 | 1.5 |
| 16 | 0.5 | 1.0 | 2.5 | 0.5 |
| 17 | 0.5 | 1.0 | 1.5 | 1.5 |
| 18 | 1.0 | 0.0 | 0.0 | 0.5 |
| 19 | 0.5 | 0.5 | 1.0 | 0.0 |
| 20 | 1.0 | 1.5 | 2.0 | 0.5 |

Having described my invention in such terms as to enable those of skill in the art to make and practice it, and having described the presently preferred embodiments thereof, I claim:

1. Apparatus for generating a filtered tremor signal representing tremor in a portion of the body of a patient, said apparatus including
(a) means for measuring tremor to generate a raw signal comprising a plurality of samples each indicating acceleration;
(b) means for filtering said raw signal to eliminate at least a portion of
(i) high frequency noise in the raw signal,
(ii) orientation,
(iii) rotation, and
(iv) voluntary motion.

2. Apparatus for generating data representing tremor in a portion of the body of a patient, said apparatus including means for
(a) measuring tremor to generate a cyclical signal comprising a plurality of samples each indicating acceleration, said signal oscillating about a selected reference line; and,
(b) examining said signal to define for each cycle comprising said cyclical signal
(i) the beginning point of said cycle,
(ii) the ending point of said cycle,
(iii) the maximum point of said cycle,
(iv) the minimum point of said cycle,
(v) the area of said cycle above said reference line, and
(vi) the area of said cycle below said reference line.

3. Apparatus for generating data representing tremor in a portion of the body of a patient, said apparatus including
(a) means for measuring tremor to generate a cyclical signal comprising a plurality of samples each indicating acceleration, said signal oscillating about a selected reference line; and,
(b) means for generating data indicating the frequency of each cycle in said cyclical signal.

4. Apparatus for generating data representing tremor in a portion of the body of a patient, said apparatus including
(a) means for measuring tremor to generate a cyclical signal comprising a plurality of samples each indicating acceleration, said signal oscillating about a selected reference line; and,
(b) means for generating data indicating the area of each cycle in said cyclical signal.

5. Apparatus for generating data representing tremor in a portion of the body of a patient, said apparatus including
(a) means for measuring tremor to generate a cyclical signal comprising a plurality of samples each indicating acceleration, said signal oscillating about a selected reference line; and,
(b) means for generating data indicating the amplitude of each cycle in said cyclical signal.

6. Apparatus for generating data representing tremor in a portion of the body of a patient, said apparatus including
(a) means for measuring tremor to generate a cyclical signal comprising a plurality of samples each indicating acceleration, said signal oscillating about a selected reference line; and,
(b) means for generating data for a selected grouping of consecutive cycles indicating at least one of a group comprising,
(i) the area of each cycle in said grouping of consecutive cycles,
(ii) the frequency of each cycle in said grouping of consecutive cycles, and
(iii) the amplitude of each cycle in said grouping of consecutive cycles.

7. Apparatus for identifying a condition associated with a patient's tremor, including
(a) means for measuring tremor to generate a cyclical signal comprising a plurality of samples each indicating acceleration;
(b) means for generating data indicating at least one characteristic of the cycles comprising said cyclical signals;
(c) means for generating a database indicating values of said characteristic for a particular condition; and,
(d) means for correlating said data with said database to determine the likelihood of the patient's tremor being associated with said condition.

8. A method for identifying a condition associated with a patient's tremor, including the steps of
(a) measuring tremor to generate a cyclical signal comprising a plurality of samples each indicating acceleration;
(b) generating data indicating at least one characteristic of the cycles comprising said cyclical signals;
(c) generating a database indicating values of said characteristic for a particular condition; and,
(d) correlating said data with said database to determine the likelihood of the patient's tremor being associated with said condition.
